(19) United States
(12) Patent Application Publication
(10) Pub. No.: US 2026/0138981 A1
(43) Pub. Date: May 21, 2026

(54) DIHYDROFOLATE REDUCTASE INHIBITORS FOR ANTI-BIOTIC RESISTANT INFECTIONS

(71) Applicant: THE TRUSTEES OF PRINCETON UNIVERSITY, Princeton, NJ (US)

(72) Inventors: Connor Chain, Princeton, NJ (US); Zemer Gitai, Princeton, NJ (US); Joseph Sheehan, Blackwood, NJ (US); Hahn Kim, Princeton, NJ (US)

(21) Appl. No.: 19/118,273

(22) PCT Filed: Oct. 6, 2023
(86) PCT No.: PCT/US2023/076235

§ 371 Date: Apr. 3, 2025

Related U.S. Application Data
(60) Provisional application No. 63/414,226, filed on Oct. 7, 2022.

(51) Int. Cl. C07D 487/04; A61K 31/198; A61K 31/513; A61K 31/519; A61K 31/635; A61K 31/708; A61P 31/04

(52) U.S. Cl. CPC C07D 487/04; A61K 31/198; A61K 31/513; A61K 31/519; A61K 31/635; A61K 31/708; A61P 31/04

(57) ABSTRACT

In one aspect, compounds and associated compositions are described herein for the treatment of various bacterial infections, including antibiotic resistant infections. In some embodiments, an antibacterial composition described herein comprises a dihydrofolate reductase inhibitor of Formula (I) and/or a salt thereof, and potentiator of the dihydrofolate reductase inhibitor of Formula (I). Moreover, in some embodiments, the antibacterial composition further comprises a thymine component, thereby enabling the antibacterial composition to selectively target bacterial species incapable of utilizing exogenous thymine.

### RELATED APPLICATION DATA

The present application claims priority pursuant to Article 8 of the Patent Cooperation Treaty to U.S. Provisional Patent Application Ser. No. 63/414,226 filed Oct. 7, 2022, which is incorporated herein by reference in its entirety.

### STATEMENT OF GOVERNMENT RIGHTS

This invention was made with government support under Grant No. DP1AI124669 awarded by the National Institutes of Health (NIH). The government has certain rights in the invention.

### FIELD

The present invention relates antibacterial compounds and, in particular, to dihydrofolate reductase inhibitors.

### BACKGROUND

The discovery of penicillin in 1929 ushered in the ‘Golden Age’ of antibiotic discovery and with it, over the next three decades, more than twenty unique classes of antibiotics. The discovery and development of these life-saving molecules has been in serious decline. Since the end of the ‘Golden Age’ in 1962 only two orally available antibiotics with completely novel targets, linezolid and a daptomycin, have been brought to the market. Declining rates of antibiotic discovery would be unalarming if it were not for evolution's perpetual offensive, constantly selecting antibiotic resistant bacteria through horizontal gene transfer and spontaneous mutation. In the United States alone, this manifests in a record 2 million antibiotic resistant infections, which annually kill 23,000 people. Moreover, such infections have been estimated to cost our health system as much as $35 billion annually. Other than better antibiotic stewardship, which has been shown to decrease the rate of hospital acquired infections, the only way to combat bacterial infections is to continuously develop antibiotics and other therapeutics presenting novel interactions and/or mechanisms of action (MOA) against various bacterial species.

### SUMMARY

In one aspect, compounds and associated compositions are described herein for the treatment of various bacterial infections, including antibiotic resistant infections. In some embodiments, an antibacterial composition described herein comprises a dihydrofolate reductase inhibitor of Formula I and/or a salt thereof:

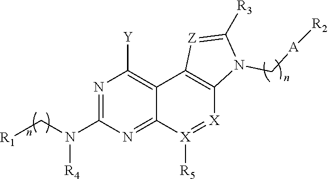

wherein R₁, R₃, R₄ and R₅ are independently selected from the group consisting of hydrogen, alkyl, alkenyl, heteroalkyl, cycloalkyl, heterocycloalkyl, aryl, heteroaryl, amide, halo, and urea, wherein the alkyl, alkenyl, heteroalkyl, cycloalkyl, heterocycloalkyl, aryl, heteroaryl, and amide are optionally substituted with one or more substituents selected from the group consisting of (C₁-C₁₀)-alkyl, (C₁-C₁₀)-alkenyl, cycloalkyl, heterocycloalkyl, aryl, heteroaryl, alkoxy, amide, sulfonamide, urea, halo, cyano, hydroxy, C(O)OR₆, and C(O)R₇, wherein R₆ is selected from the group consisting of hydrogen, alkyl and alkenyl and R₇ is selected from the group consisting of hydrogen, alkyl, alkenyl, heteroalkyl, cycloalkyl, heterocycloalkyl, aryl, heteroaryl and NR₈R₉, wherein R₈ and R₉ are independently selected from the group consisting of hydrogen, alkyl, alkenyl, aryl and heteroaryl; and
wherein R₂ is selected from the group consisting of halo, fluoroalkyl, and cyano; and
wherein A is heteroarylene; and
wherein X and Z are independently selected from the group consisting of C, N, O and S; and
wherein Y is selected from the group consisting of hydrogen, OH, alkoxy, and NR₁₀R₁₁, wherein R₁₀ and R₁₁ are independently selected from the group consisting of hydrogen, alkyl, heteroalkyl, cycloalkyl, heterocycloalkyl, alkenyl, aryl, heteroaryl, amide, sulfonamide, urea, alkylene-aryl, alkylene-heteroaryl, and C(O)R₁₂ wherein R₁₂ is selected from the group consisting of hydrogen, alkyl, alkenyl, heteroalkyl, cycloalkyl, heterocycloalkyl, aryl and heteroaryl, and wherein R₁₀ and R₁₁ may optionally form a ring structure, wherein the aryl, heteroaryl, alkylene-aryl and alkylene heteroaryl are optionally substituted with one or more substituents selected from the group consisting of alkyl, alkenyl, alkynyl, halo, and alkynylene-alkylsilane; and n is an integer from 0 to 5; and
and a potentiator of the dihydrofolate reductase inhibitor of Formula I and/or salt thereof. In some embodiments, for example, A is pyrinidylene, and R₂ is selected from the group consisting of halo and fluoroalkyl. Alternatively, A is thiophenylene, and R₂ is cyano.

In another aspect, an antibacterial composition described herein comprises a dihydrofolate reductase inhibitor of Formula I and/or a salt thereof:

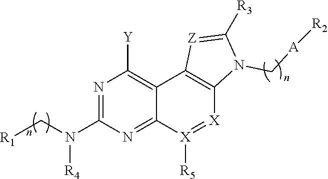

wherein R₁, R₃, R₄ and R₅ are independently selected from the group consisting of hydrogen, alkyl, alkenyl, heteroalkyl, cycloalkyl, heterocycloalkyl, aryl, heteroaryl, amide, halo, and urea, wherein the alkyl, alkenyl, heteroalkyl, cycloalkyl, heterocycloalkyl, aryl, heteroaryl, and amide are optionally substituted with one or more substituents selected from the group consisting of (C₁-C₁₀)-alkyl, (C₁-C₁₀)-alkenyl, cycloalkyl, heterocycloalkyl, aryl, heteroaryl, alkoxy, amide, sulfonamide, urea, halo, cyano, hydroxy, C(O)OR₆, and C(O)R₇, wherein R₆ is selected from the group consisting of hydrogen, alkyl and alkenyl and R₇ is selected from the group consisting of hydrogen, alkyl, alkenyl, heteroalkyl, cycloalkyl, heterocycloalkyl, aryl, heteroaryl and NR₈R₉, wherein R₈ and R₉ are independently selected from the group consisting of hydrogen, alkyl, alkenyl, aryl and heteroaryl; and
wherein R₂ is selected from the group consisting of halo, fluoroalkyl, and cyano; and
wherein A is heteroarylene; and
wherein X and Z are independently selected from the group consisting of C, N, O and S; and
wherein Y is selected from the group consisting of hydrogen, OH, alkoxy, and NR₁₀R₁₁, wherein R₁₀ and R₁₁ are independently selected from the group consisting of hydrogen, alkyl, heteroalkyl, cycloalkyl, heterocycloalkyl, alkenyl, aryl, heteroaryl, amide, sulfonamide, urea, alkylene-aryl, alkylene-heteroaryl, and C(O)R₁₂ wherein R₁₂ is selected from the group consisting of hydrogen, alkyl, alkenyl, heteroalkyl, cycloalkyl, heterocycloalkyl, aryl and heteroaryl, and wherein R₁₀ and R₁₁ may optionally form a ring structure, wherein the aryl, heteroaryl, alkylene-aryl and alkylene heteroaryl are optionally substituted with one or more substituents selected from the group consisting of alkyl, alkenyl, alkynyl, halo, and alkynylene-alkylsilane; and n is an integer from 0 to 5; and
a thymine component.
   In some embodiments, a potentiator of the dihydrofolate reductase inhibitor of Formula I and/or salt thereof is present in addition to the thymine component.

As described further herein, presence of the thymine component can be employed to selectively target bacteria incapable of utilizing exogenous thymine. The presence of the thymine component, for example, can rescue bacteria from the deleterious effects of dihydrofolate reductase inhibitors of Formula I and/or salts thereof, provided that such bacteria exhibit thymidine kinase and thymidine phosphorylase activity for processing exogenous thymine. Accordingly, bacterial species lacking thymidine kinase and thymidine phosphorylase activity cannot be rescued and are thereby selectively targeted for destruction by dihydrofolate reductase inhibitor of Formula I and/or salts thereof. In some embodiments, presence of the thymine component facilitates selective targeting of P. aeruginosa.

In another aspect, methods of treating bacterial infections are described herein. In some embodiments, a method comprises treating an infection of pathogenic bacteria by administering to a patient in need thereof a therapeutically effective amount of a composition comprising a dihydrofolate reductase inhibitor of Formula I and/or a salt thereof:

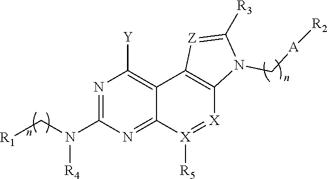

wherein R₁, R₃, R₄ and R₅ are independently selected from the group consisting of hydrogen, alkyl, alkenyl, heteroalkyl, cycloalkyl, heterocycloalkyl, aryl, heteroaryl, amide, halo, and urea, wherein the alkyl, alkenyl, heteroalkyl, cycloalkyl, heterocycloalkyl, aryl, heteroaryl, and amide are optionally substituted with one or more substituents selected from the group consisting of (C₁-C₁₀)-alkyl, (C₁-C₁₀)-alkenyl, cycloalkyl, heterocycloalkyl, aryl, heteroaryl, alkoxy, amide, sulfonamide, urea, halo, cyano, hydroxy, C(O)OR₆, and C(O)R₇, wherein R₆ is selected from the group consisting of hydrogen, alkyl and alkenyl and R₇ is selected from the group consisting of hydrogen, alkyl, alkenyl, heteroalkyl, cycloalkyl, heterocycloalkyl, aryl, heteroaryl and NR₈R₉, wherein R₈ and R₉ are independently selected from the group consisting of hydrogen, alkyl, alkenyl, aryl and heteroaryl; and
wherein R₂ is selected from the group consisting of halo, fluoroalkyl, and cyano; and
wherein A is heteroarylene; and
wherein X and Z are independently selected from the group consisting of C, N, O and S; and
wherein Y is selected from the group consisting of hydrogen, OH, alkoxy, and NR₁₀R₁₁, wherein R₁₀ and R₁₁ are independently selected from the group consisting of hydrogen, alkyl, heteroalkyl, cycloalkyl, heterocycloalkyl, alkenyl, aryl, heteroaryl, amide, sulfonamide, urea, alkylene-aryl, alkylene-heteroaryl, and C(O)R₁₂ wherein R₁₂ is selected from the group consisting of hydrogen, alkyl, alkenyl, heteroalkyl, cycloalkyl, heterocycloalkyl, aryl and heteroaryl, and wherein R₁₀ and R₁₁ may optionally form a ring structure, wherein the aryl, heteroaryl, alkylene-aryl and alkylene heteroaryl are optionally substituted with one or more substituents selected from the group consisting of alkyl, alkenyl, alkynyl, halo, and alkynylene-alkylsilane; and n is an integer from 0 to 5; and
administrating to the patient a potentiator of the dihydrofolate reductase inhibitor of Formula I and/or salt thereof.

As described above, a thymine component can be administered to the patient for selectively targeting bacteria incapable of utilizing exogenous thymine. The thymine component can be part of the composition comprising the dihydrofolate reductase inhibitor of Formula I and/or a salt thereof. Alternatively, the thymine component can be administered to the patient as a composition separate or independent of the dihydrofolate reductase inhibitor of Formula I. In some embodiments, a potentiator is not part of the antibacterial composition when thymine is employed.

These and other embodiments are further described in the following detailed description.

### BRIEF DESCRIPTION OF THE DRAWINGS

FIG. 1A is a schematic of folate metabolism in PA14, grey crossed circles indicate enzymes lacking activity in PA14.

FIG. 1B provides the structure of fluorofolin.

FIG. 1C quantifies colony forming units (CFU/mL) of P. aeruginosa PA14 after 4-hour treatment with 5% DMSO (solvent control), 6.2 μg/mL fluorofolin (2×MIC), 250 μg/mL trimethoprim (2×MIC), or 4 μg/mL polymixin B (2×MIC). Data points represent 3 biological replicates with 3 technical replicates. Mean±SD are shown.

FIG. 1D provides DHFR (FolA) activity measured on purified E. coli FolA through measuring the change in sample absorbance at 340 nm due to DHFR-dependent NADPH consumption. Activity was related to an untreated standard condition using 60 M NADPH and 100 μM DHF. IC50 values were derived from the Hill equation fits on reactions performed with increasing antibiotic concentrations.

FIG. 1E is an analogous assay to FIG. 2B using purified human DHFR.

FIG. 1F provides metabolite abundance of deoxyuridine monophosphate (dUMP), aminoimidazole carboxamide ribotide (AICAR), and glycinamide ribonucleotide (GAR) of P. Aeruginosa PA14 treated with 5% DMSO (solvent control), 6.3 μg/mL fluorofolin (2×MIC) or 250 μg/mL trimethoprim (2×MIC) for 15 min. Metabolite abundance was quantified in comparison to the solvent only control. Data represents mean±SD for 3 biological replicates. P values were calculated using unpaired t-test using Prism 9 (P values≤0.05)

FIG. 1G provides intrabacterial drug metabolism (IBDM) of PA14 treated with 5 μM fluorofolin or trimethoprim. Samples were taken at time t=0, 30, 60, and 90 minutes. Data represent mean±SD of triplicate technical replicates.

FIG. 1H provides cumulative accumulation of drugs over 90 mins by AUC of IBDM curves. Data represent mean±SD of triplicate technical replicates. (P value≤0.0001) t-test using Prism 9.

FIG. 1I is the minimum inhibitory concentration of fluorofolin or trimethoprim against transposon mutants for each component of the MexAB-OprM efflux pump. MIC against wildtype PA14 was calculated to control for each drug stock. MIC values are representative of two independent replicates.

FIG. 2A quantifies plasma concentration of fluorofolin over time after single oral administration to neutropenic CD1 mice. Each line is representative of an individual mouse.

FIG. 2B is a Checkerboard assay of fluorofolin and sulfamethoxazole. Z-values represent fractional inhibitory concentrations (FICs). FICs were determined by dividing the MIC of each drug when used in combination by the MIC when used alone. FIC of less than or equal to 0.5 is considered a synergistic effect.

FIG. 2C quantifies treatment of mice with fluorofolin (SC) with or without SMX 100 mg/kg (IP).

FIG. 2D characterizes Fluorofolin and SMX treatment of mice fed a diet of thymidine-supplemented chow during PA14 infection. Mice were treated 1 and 12 hours post infection (n=5 for each group). P values from Tukey's multiple comparisons test (P-values≤0.0001).

FIGS. 3A and 3B characterize the growth of E. coli MG1655 and P. aeruginosa PA14 respectively in 0.3 mM thymine, methionine, and inosine (TMI) supplemented media and treated with fluorofolin at 2×MIC or DMSO. Curves represent optical at 600 nm (OD₆₀₀) of 2 biological replicates. Mean±SD are shown.

FIG. 3C characterizes competition of P. aeruginosa PA14 and E. coli MG1655 in LB or TMI-supplemented LB media. PA14 and E. coli MG1655 were inoculated at a 1:1 ratio and grown overnight in the presence or absence of 50 μg/mL fluorofolin in each media condition. The following day CFU were counted on LB-agar or Pseudomonas selection agar plates to determine CFU/mL of each species. Data represent mean±SD of triplicate biological and triplicate technical replicates. P values were calculated using unpaired t-test using Prism 9 (P value≤0.001)

FIG. 4A provides RNA sequencing results from nfxB (T39P) mutant expression of efflux pump proteins relative to wildtype PA14.

FIG. 4B illustrates nfxB (T39P) mutants showing cross resistance to both ciprofloxacin (2×MIC) and fluorofolin (2×MIC).

FIG. 4C provides RNA sequencing results from mexS (L46F) mutant expression of efflux pump proteins relative to wildtype PA14.

FIG. 4D quantifies pyocyanin production of nfxB (T39P) and mexS (L46F) mutants. Pyocyanin levels were measured through integration of absorbances from 306-326 nm. A ΔpqsA PA14 mutant was included as this strain does not make pyocyanin and ΔpqsA absorbance values were used to subtract out background signal. 1 μM PQS in DMSO was added to samples at inoculation to rescue pyocyanin production. P values were calculated using unpaired t-test using Prism 9 (****P value<0.0001, ***P value<0.001).

FIG. 4E illustrates C. elegans N2 toxicity after infection with wildtype PA14 (n=158), nfxB T39P (n=131) or mexS L46F (n=128) was measured over 60 hours. Worms were declared dead if they lacked movement after gently poking with forceps. P values were calculated using a Mantel-Cox test compared to wildtype PA14 from Prism 9.

FIG. 4F characterizes clinical isolate resistance to fluorofolin and ciprofloxacin was tested by treating the panel with 50 μg/mL of either antibiotic. Growth inhibition was determined by comparing OD600 after 16 hours to DMSO treated controls. Strains with growth inhibition>80% were considered sensitive to fluorofolin treatment.

FIG. 5A provides flow cytometry results of PA14 stained with the membrane permeability dye TO-PRO-3. Cells were incubated for 15 min with 5% DMSO (solvent control), 4 μg/mL polymixin B (2×MIC), 250 μg/mL trimethoprim (2×MIC), or 6.25 μg/mL fluorofolin (2×MIC). The gates were determined for TO-PRO-3 staining using solvent only and polymixin B controls.

FIG. 5B provides flow cytometry of E. coli lptD4213 treated with 5% DMSO, 5 μM CCCP, or 2×MIC fluorofolin 0.04 μg/mL polymixin B, and 0.4 μg/mL trimethoprim and stained with TO-PRO-3 and DiOC2(3).

FIG. 5C shows hemolysis of 6×10⁶ sheep red blood cells after treatment with selected antibiotics for 1 hour. Percent hemolysis was measured using Abs₄₀₅ compared to 100% lysis control by Triton X-100 (1% v/v). Mean±SD of technical triplicates are shown.

FIG. 5D provides IC50 of fluorofolin against in vitro mammalian cell lines relative to the IC50 of IRS-16. HLF: human lung fibroblast, HK-2: human kidney epithelial, PBMC: peripheral blood mononuclear cell, WI-38: Embryonic lung tissue.

FIGS. 6A-6E illustrate growth of various bacterial species with or without TMI supplementation (0.3 mM thymine, methionine, and inosine) and treated with fluorofolin at 2× MIC or DMSO.

FIG. 6F quantifies P. aeruginosa PA14 or E. coli lptd4213 treated with fluorofolin 50 μg/mL in the presence or absence of 0.3 mM thymidine.

FIG. 6G quantifies E. coli lptd4213 treated with fluorofolin 50 μg/mL and SMX (78.1 μg/mL) in the presence or absence of 0.3 mM thymidine or 0.3 mM TMI supplementation, Curves represent optical at 600 nm (OD₆₀₀) of 2 biological replicates. Mean±SD are shown.

### DETAILED DESCRIPTION

Embodiments described herein can be understood more readily by reference to the following detailed description and examples and their previous and following descriptions. Elements, apparatus and methods described herein, however, are not limited to the specific embodiments presented in the detailed description and examples. It should be recognized that these embodiments are merely illustrative of the principles of the present invention. Numerous modifications and adaptations will be readily apparent to those of skill in the art without departing from the spirit and scope of the invention.

### Definitions

The term “alkyl” as used herein, alone or in combination, refers to a straight or branched saturated hydrocarbon group optionally substituted with one or more substituents. For example, an alkyl can be C₁-C₃₀ or C₁-C₁₈.

The term “alkenyl” as used herein, alone or in combination, refers to a straight or branched chain hydrocarbon group having at least one carbon-carbon double bond and optionally substituted with one or more substituents

The term “alkynyl” as used herein, alone or in combination, refers to a straight or branched chain hydrocarbon group having at least one carbon-carbon triple bond and optionally substituted with one or more substituents including, but not limited to, alkyl, cycloalkyl, heterocycloalkyl, aryl, heteroaryl, amine, and/or alkylsilane.

The term “aryl” as used herein, alone or in combination, refers to an aromatic monocyclic or multicyclic ring system optionally substituted with one or more ring substituents.

The term “heteroaryl” as used herein, alone or in combination, refers to an aromatic monocyclic or multicyclic ring system in which one or more of the ring atoms is an element other than carbon, such as nitrogen, oxygen and/or sulfur.

The term “cycloalkyl” as used herein, alone or in combination, refers to a non-aromatic, mono- or multicyclic ring system optionally substituted with one or more ring substituents.

The term “heterocycloalkyl” as used herein, alone or in combination, refers to a non-aromatic, mono- or multicyclic ring system in which one or more of the atoms in the ring system is an element other than carbon, such as nitrogen, oxygen or sulfur, alone or in combination, and wherein the ring system is optionally substituted with one or more ring substituents.

The term “heteroalkyl” as used herein, alone or in combination, refers to an alkyl moiety as defined above, having one or more carbon atoms in the chain, for example one, two or three carbon atoms, replaced with one or more heteroatoms, which may be the same or different, where the point of attachment to the remainder of the molecule is through a carbon atom of the heteroalkyl radical.

The term “alkoxy” as used herein, alone or in combination, refers to the moiety RO—, where R is alkyl or alkenyl defined above.

The term “halo” as used herein, alone or in combination, refers to elements of Group VIIA of the Periodic Table (halogens). Depending on chemical environment, halo can be in a neutral or anionic state. Halo, for example, includes fluoro, chloro, bromo, and iodo.

### I. Antibacterial Compositions

As provided above, antibacterial compositions described herein comprise dihydrofolate reductase inhibitors of Formula I and/or salts thereof in conjunction with potentiators of the dihydrofolate reductase inhibitors. Dihydrofolate reductase inhibitor of Formula I and/or a salt thereof can be present in a composition described herein in any amount consistent with treating bacterial infections, such as gram negative bacterial infections. In some embodiments, a dihydrofolate reductase inhibitor of Formula I and/or salt thereof is present in an amount or concentration of 0.001 μg/ml to 1 mg/ml. Dihydrofolate reductase inhibitors of Formula I and/or salts thereof can also be present in an amount or concentration selected from Table I.

TABLE I
Amount of Compound of Formula I (μg/ml)
0.001-100
0.001-10
0.01-50
0.05-30
0.1-10

The amount or concentration of a dihydrofolate reductase inhibitor of Formula I and/or salt thereof in compositions described herein can be dependent on the identity and/or nature of the bacteria being treated and/or the efficacy of the potentiator included in the composition.

In some embodiments, a dihydrofolate reductase inhibitor of Formula I is selected from the group consisting of one of the following structures:

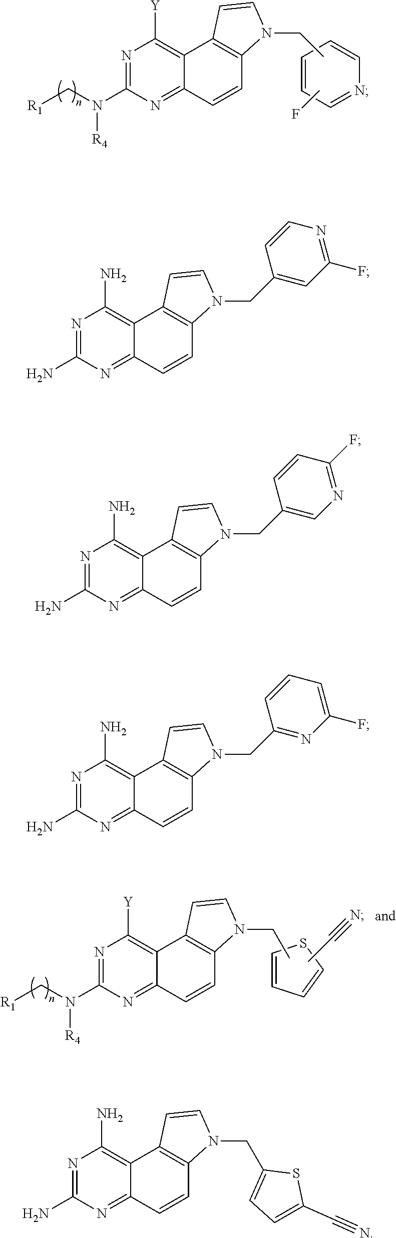

Antibacterial compositions described herein can also comprise a potentiator of the dihydrofolate reductase inhibitor of Formula I and/or salt thereof. Any potentiator consistent with the technical objectives described herein can be employed. In some embodiments, suitable potentiators include one or more antibacterial compounds, including sulfonamide compounds. Potentiator of an antibacterial composition described herein, for example, can comprise sulfamethoxazole, in some embodiments. Potentiator can be present in antibacterial compositions described herein in any desired amount. The amount of potentiator can be dependent on several considerations including, but not limited to, the specific identity and/or amount of the dihydrofolate reductase inhibitor of Formula I and/or salt thereof, and the identity of the bacterial species being treated with the composition. In some embodiments, potentiator is present in the antibacterial composition in an amount or concentration of 50 μg/mL to 300 μg/mL or 100 μg/mL to 200 μg/mL. In some embodiments, the potentiator is administered with the dihydrofolate reductase inhibitor of Formula I and/or salt thereof as a single mixture. Alternatively, the potentiator and the dihydrofolate reductase inhibitor of Formula I and/or salt thereof are administered to the patient separately or independently. When separate, the potentiator and the dihydrofolate reductase inhibitor of Formula I and/or salt thereof can be administered simultaneously or sequentially in any desired order. Moreover, the dihydrofolate reductase inhibitor of Formula I and potentiator can be delivered via different mechanisms. In some embodiments, for example, the dihydrofolate reductase inhibitor of Formula I is administered subcuntaneously, and the potentiator is administered intraperitoneally.

Moreover, a dihydrofolate reductase inhibitor of Formula I and/or salt thereof in the absence of the potentiator can exhibit a minimum inhibitory concentration (MIC) for a bacterial species/strain less than 5 μg/ml or less than 1 μg/ml, in some embodiments. MIC of the dihydrofolate reductase inhibitor of Formula I and/or salt thereof can be further reduced in the presence of the potentiator. Presence of the potentiator, in some embodiments, can reduce the MIC by an order of magnitude. For example, a dihydrofolate reductase inhibitor of Formula I and/or salt thereof in the presence of potentiator can exhibit a MIC of 0.1-0.5 μg/ml for P. aeruginosa, in some embodiments.

Additionally, a dihydrofolate reductase inhibitor of Formula I and/or salt thereof, in some embodiments, does not induce membrane depolarization and/or permeabilization of bacterial cells. Lack of membrane depolarization and/or permeabilization can limit damage to eukaryotic cells during patient treatment with compositions described herein.

In another aspect, bacterial compositions described herein also comprise a thymine component in addition to the dihydrofolate reductase inhibitor of Formula I and/or salt thereof. Presence of the thymine component can be employed to selectively target bacteria incapable of utilizing exogenous thymine. Presence of the thymine component, for example, can rescue bacteria from the deleterious effects of dihydrofolate reductase inhibitors of Formula I and/or salts thereof, provided that such bacteria exhibit thymidine kinase and thymidine phosphorylase activity for processing exogenous thymine. Accordingly, bacterial species lacking thymidine kinase and thymidine phosphorylase activity cannot be rescued and are thereby selectively targeted for destruction by dihydrofolate reductase inhibitor of Formula I and/or salts thereof. In some embodiments, presence of the thymine component facilitates selective targeting of P. aeruginosa, as shown in the examples herein. In some embodiments, the thymine component comprises thymine, methionine, and/or inosine (TMI). The thymine component can be present in any amount consistent with the technical objectives described herein. The amount of thymine component, for example, can be dependent on several considerations including identity and/or amount of the dihydrofolate reductase inhibitor of Formula I and/or salt thereof, and the identity or identities of bacterial species not being targeted with the dihydrofolate reductase inhibitor of Formula L In some embodiments, the thymine component is present or administered in an amount of 0.5 g/kg to 5 g/kg. The thymine component can be employed in an antibacterial composition in the absence of the potentiator, in some embodiments. Alternatively, the thymine component and potentiator can be employed in the same antibacterial composition comprising the dihydrofolate reductase inhibitor of Formula I and/or salt thereof.

In some embodiments, the thymine component is co-administered with the dihydrofolate reductase inhibitor of Formula I and/or potentiator. Such co-administration can be in a single composition or via multiple independent compositions. Alternatively, the thymine component can be administered at a time period before or after administration of the dihydrofolate reductase inhibitor of Formula I and/or potentiator.

### II. Methods of Treating Bacterial Infections

In another aspect, methods of treating bacterial infections are provided herein. In some embodiments, a method comprises treating an infection of pathogenic bacteria, including gram negative bacteria, by administering to a patient in need thereof a therapeutically effective amount of a composition comprising a dihydrofolate reductase inhibitor of Formula I and/or salt thereof, and administering a potentiator of the dihydrofolate reductase, and/or a thymine component. Compositions comprising the dihydrofolate reductase inhibitor of Formula I and/or salt thereof can have any properties and/or characteristics described in Section I hereinabove. The dihydrofolate reductase inhibitor of Formula I and/or salt thereof, for example, can be present in an amount or concentration selected from Table I above. Moreover, the bacterial composition can be combined with any physiologically acceptable excipient for administration to the patient. In some embodiments, pathogenic bacteria treated according to methods described herein include P. aeruginosa.

These and other embodiments are further illustrated in the following non-limiting examples.

### Examples—Compositions Inhibiting P. aeruginosa

The gram-negative opportunistic pathogen, Pseudomonas aeruginosa, is of particular interest for antibiotic development as it has evolved multiple mechanisms to evade antibiotics including a robust outer membrane multiple efflux pumps, and other antibiotic resistance determinants like carbapenamases. P. aeruginosa is often associated with chronic infections and the resulting prolonged exposure to antibiotics can have detrimental health effects due to microbiome disruption. Beyond the problem that most antibiotics do not exhibit significant efficacy against P. aeruginosa, there are no commercial narrow-spectrum antibiotics that selectively target P. aeruginosa.

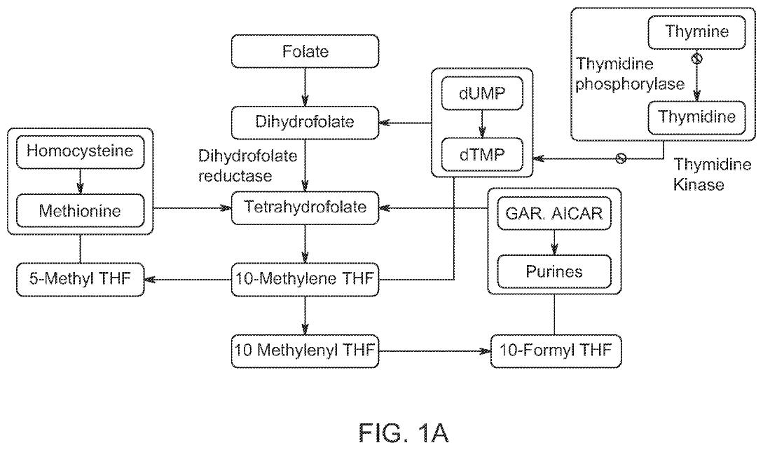

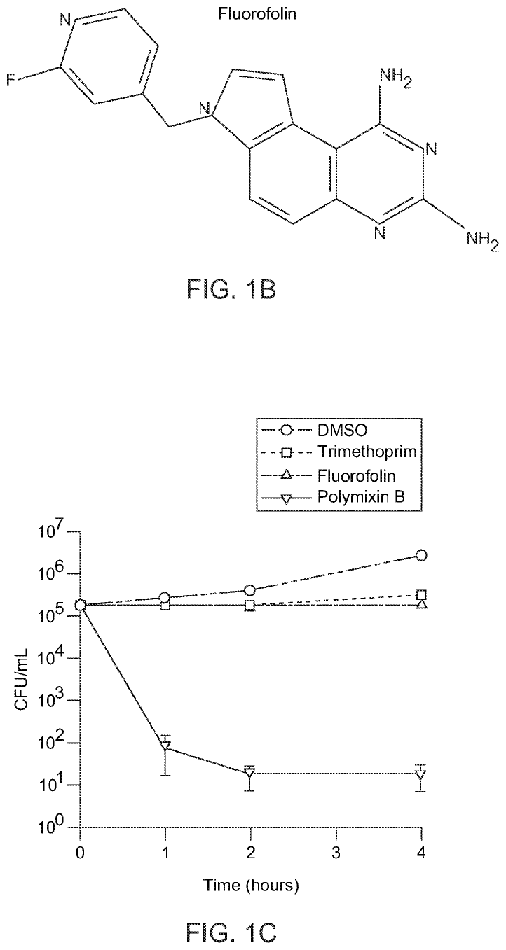

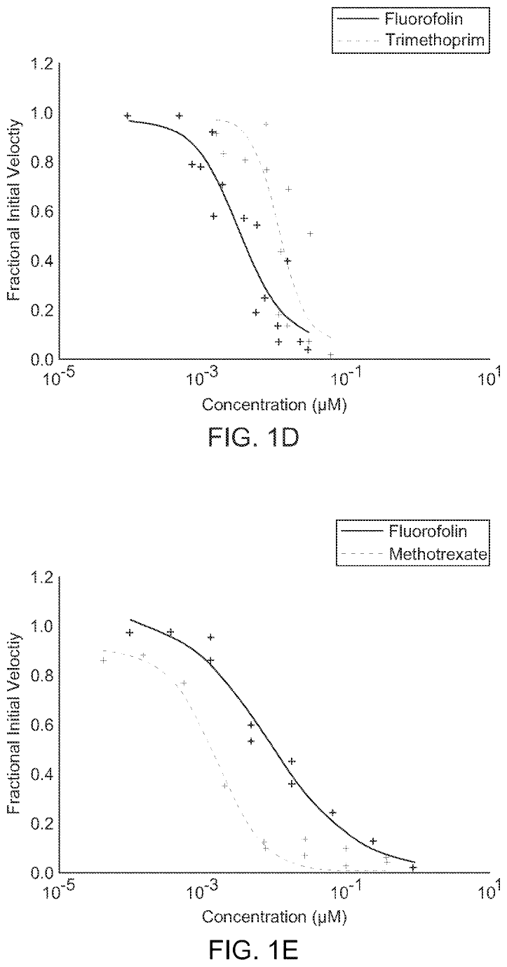

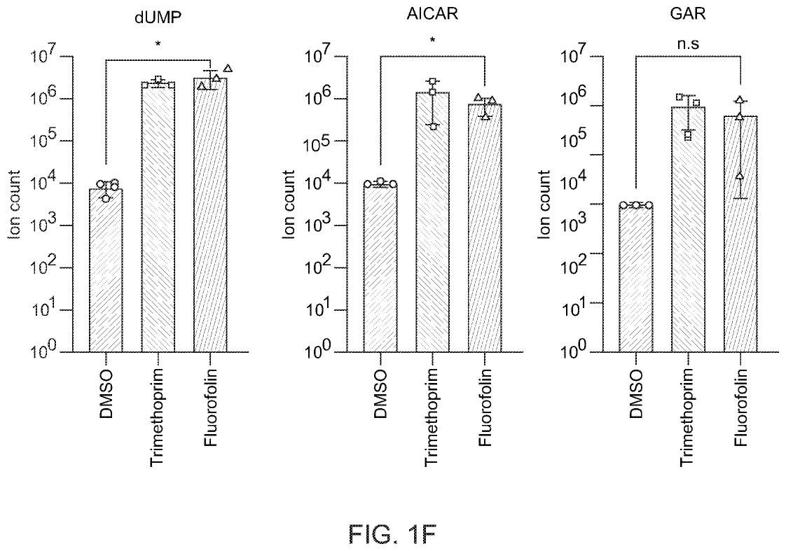

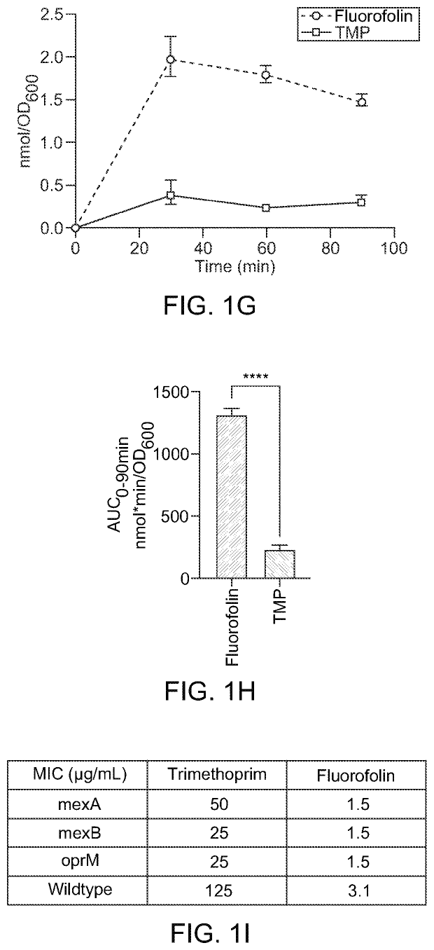

There is mounting evidence for the potential benefits of antibiotics with narrow species selectivity, as they can target pathogens of interest with minimal disruption to the host microbiome. Existing strategies for narrow-spectrum targeting have largely focused on developing drugs whose targets are only present in specific pathogens. However, there are also species-specific genetic and metabolic differences that could hypothetically be exploited to cause antibiotics with widely conserved molecular targets to only kill specific species of interest. In particular, unlike most enteric bacteria, P. aeruginosa lacks thymidine kinase and thymidine phosphorylase activity, and is therefore unable to scavenge thymine from the environment (FIG. 1A). It was therefore hypothesized that targeting de novo thymidylate synthesis with concurrent thymine supplementation could selectively target P. aeruginosa.

Dihydrofolate reductase (DHFR) catalyzes the production of tetrahydrofolate, which is required for de novo thymidylate synthesis. There are known DHFR inhibitors, such as the commercially-used antibiotic trimethoprim (TMP). However, P. aeruginosa is intrinsically resistant to all known DHFR inhibitors, including TMP. In the present example, a new DHFR inhibitor, fluorofolin, is characterized that shows potent activity against P. aeruginosa in vitro and in a mouse model. In being a dihydrofolate reductase inhibitor of Formula I, fluorofolin is of the formula:

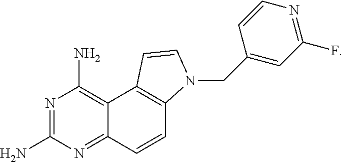 [SMILES: Nc1nc(N)c2c(ccc3c2ccn3Cc2ccnc(F)c2)n1]

Fluorofolin is employed to both selectively eliminate P. aeruginosa from mixed species bacterial cultures and to uncover a trade-off between P. aeruginosa antibiotic resistance and virulence. These studies demonstrate the utility of inhibitors of broadly conserved targets as both narrow-spectrum agents and tools for discovery of context-specific features of pathogenesis.

Fluorofolin alone exhibited potent activity against P. aeruginosa PA14 (MIC=3.1 μg/mL). Fluorofolin also provided broad spectrum antibiotic activity, as it was also capable of inhibiting growth of two other strains of P. aeruginosa, PA01 and ATTC 27853, as well as all 5 of the ESKAPE pathogens tested at concentrations less than 50 μg/mL (Table 2).

TABLE 2
Minimum Inhibitory Concentration (MIC) of
fluorofolin against a panel of bacteria.

| Species | Strain | MIC (ug/mL) |
|---|---|---|
| E. faecalis | ATCC 51575* | 0.01 |
| E. coli | lptD4213 | 0.02 |
| S. typhimurium | CMCC 50115* | 0.03 |
| S. aureus | NRS384* | 0.03 |
| E. coli | NCM-3722 | 0.3125 |
| S. epidermidis | EGM-206 | 0.3125 |
| S. aureus | USA300 | 0.4 |
| E. coli | MG1655 | 0.625 |
| A. baumannii | ATCC 17978* | 0.8 |
| P. aeruginosa | PA14 | 3.125 |
| P. aeruginosa | PA01 | 6.25 |
| K. pneumoniae | ATCC 438165 | 6.25 |
| E. coli | ATCC BAA-198* | 7.11 |
| E. cloacae | ATCC BAA-1143 | 12.5 |
| P. aeruginosa | ATTC 27853 | 25 |
| A. baumannii | BAA-125 | 31.25 |

MIC represents the concentration of fluorofolin at which no bacterial growth is detected after 16 hours at 37° C. in LB Broth as measured by OD₆₀₀. MIC of strains denoted with * were performed by WuXi in Mueller-Hinton Broth. MIC values were calculated from duplicate samples.

Fluorofolin exhibited bacteriostatic activity in rich media (FIG. 1C). Based on this finding, the mechanism of action (MoA) of fluorofolin was further investigated. The ability of fluorofolin to directly inhibit the enzymatic activity of purified E. coli DHFR (FolA) was first examined. Fluorofolin inhibited DHFR activity with an IC50 of 2.5±1.1 nM, which was comparable to that of TMP (IC50 of 8.7±3.6 nM) (FIG. 1D). Fluorofolin also exhibited modest specificity for bacterial DHFR in vitro; in an analogous assay using purified human DHFR, fluorofolin had an IC50 of 14.0±4 nM (FIG. 1E).

To test the ability of fluorofolin to inhibit P. aeruginosa DHFR in vivo, metabolomics were performed. As a positive control for DHFR inhibition, we turned to TMP. P. aeruginosa is resistant to TMP at the highest doses tolerated by humans and is thus not clinically useful in targeting P. aeruginosa infections, but it was found that TMP can inhibit the growth of P. aeruginosa cultures at very high concentrations in vitro (PA14 MIC=125 μg/mL, approximately 70 times higher than the mean steady state serum concentration of TMP achievable in serum after clinical use of co-trimoxazole). Metabolomics demonstrated that both TMP and fluorofolin treatment caused significant upregulation of the purine and dTTP intermediates 5-aminoimidazole-4-carboxamide ribonucleotide (AICAR) and 2′-deoxyuridine 5′-monophosphate (dUMP) in P. aeruginosa (P=0.0186 and 0.0196 respectively) (FIG. 1F). These results support the conclusion that fluorofolin targets folate metabolism in P. aeruginosa.

To predict how fluorofolin might interact with P. aeruginosa DHFR, molecular docking predictions were performed. As the structure of P. aeruginosa DHFR has not been solved, AlphaFold was used to generate a homology model for its three-dimensional structure based on its amino acid sequence. The NADPH cofactor was inserted into this structure and aligned it to the experimentally-derived structure of NADPH and trimethoprim bound to E. coli DHFR. AutoDock Vina was the used to predict the binding of P. aeruginosa DHFR to either fluorofolin or trimethoprim. Both fluorofolin and trimethoprim were predicted to bind DHFR in the dihydrofolate binding pocket and fluorofolin was predicted to have a lower binding affinity (−9.104 kcal/mol) than trimethoprim (−6.8 kcal/mol). The stronger binding of fluorofolin may be explained by an additional hydrogen bond between Leu23 of P. aeruginosa DHFR and the pyridine group of fluorofolin that is not formed with trimethoprim.

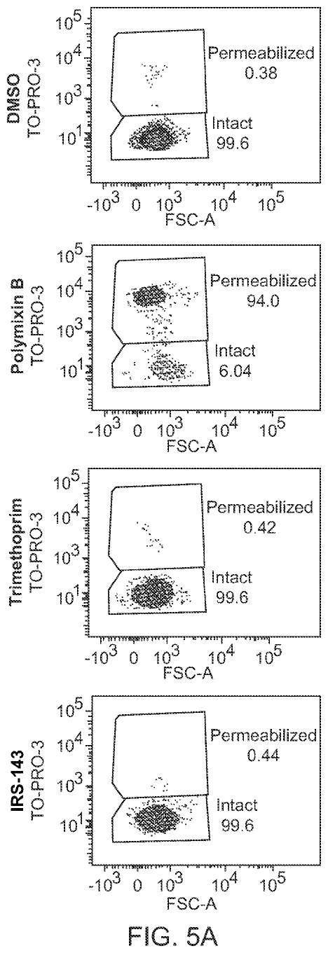

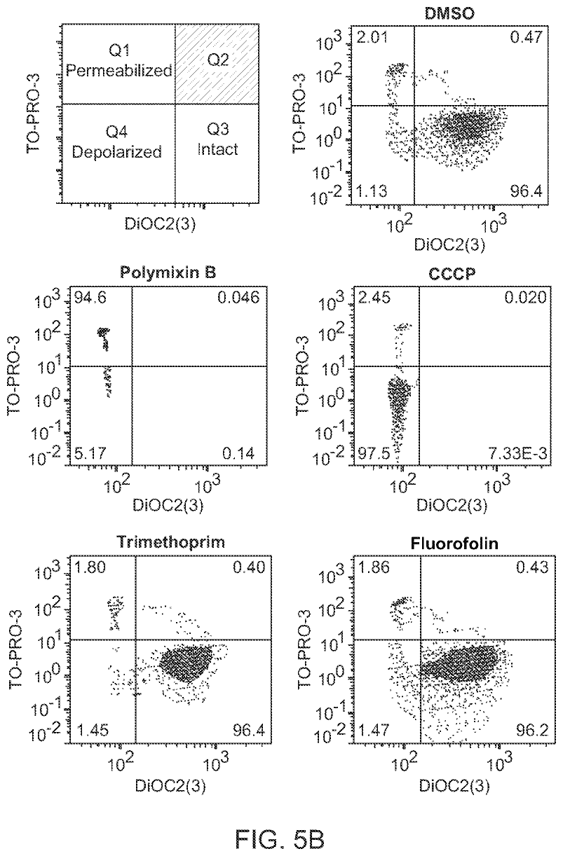

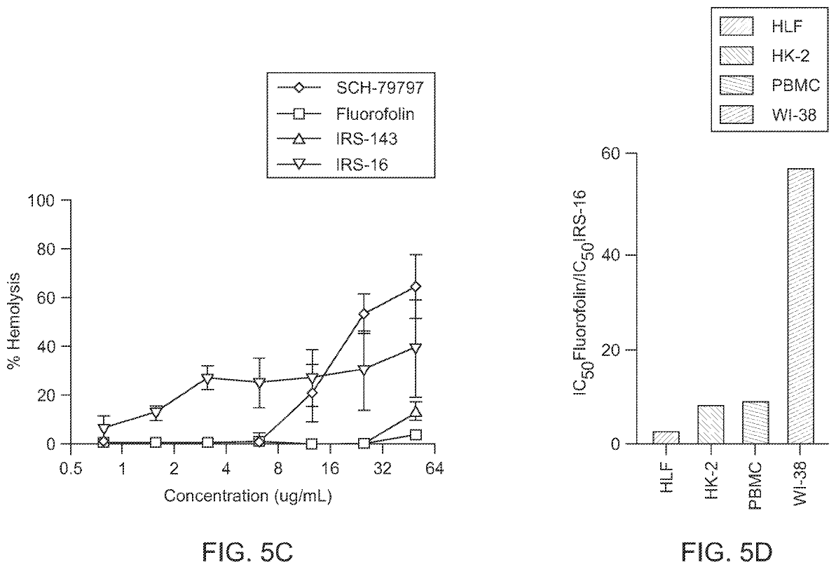

The ability of fluorofolin to permeabilize P. aeruginosa PA14 was examined. Specifically, flow cytometry of P. aeruginosa stained with TO-PRO-3 was used, treated the bacteria with 2×MIC, and analyzed membrane integrity. It was observed that fluorofolin does not cause significant disruption of P. aeruginosa PA14 membranes (FIG. 5A). It was also confirmed that fluorofolin does not cause membrane depolarization or permeabilization in E. coli lptD4213 (FIG. 5B, the polarization assay could not be performed in P. aeruginosa as the membrane polarization reporter DiOC2(3) penetrates the outer membrane of E. coli but not P. aeruginosa). It was hypothesized that this decrease in membrane targeting might also decrease toxicity due to the disruption of eukaryotic membranes. As predicted, fluorofolin did not cause significant permeabilization of red blood cell membranes at biologically relevant concentrations (FIG. 5C) and displayed lower toxicity towards several mammalian cell types including a 9.6-fold increase in IC50 against PBMCs and a 58.5-fold increase in IC50 against WI-38 cells compared to IRS-16 (FIG. 5D).

As fluorofolin and TMP exhibit similar functional inhibition of purified DHFR, it was investigated why fluorofolin was better at inhibiting P. aeruginosa growth. It was hypothesized that fluorofolin may better accumulate inside of P. aeruginosa. P. aeruginosa is particularly drug resistant due to its robust outer membrane and expression of multiple RND-type efflux pumps. Using mass spectrometry to measure drug accumulation, it was found that fluorofolin accumulated in P. aeruginosa more rapidly (FIG. 1G) and to higher levels (FIG. 1H) than TMP. It was previously shown that the constitutively active efflux pump MexAB-OprM can export TMP. It was confirmed that PA14 mutants with disruptions in mexA, mexB and oprM showed large reductions in MIC to TMP (FIG. 1I). However, these mutants did not decrease their MIC against fluorofolin to the same extent (FIG. 1I), suggesting that the increased accumulation of fluorofolin is in part due to decreased efflux of fluorofolin by MexAB-OprM. It was also noted TMP still has a higher MIC than fluorofolin in mexA, mexB and oprM mutants, suggesting that fluorofolin may also improve other features of cell accumulation such as membrane penetrance or interactions with other efflux pumps.

Fluorofolin Inhibits P. aeruginosa PA14 in a Murine Thigh Infection Model

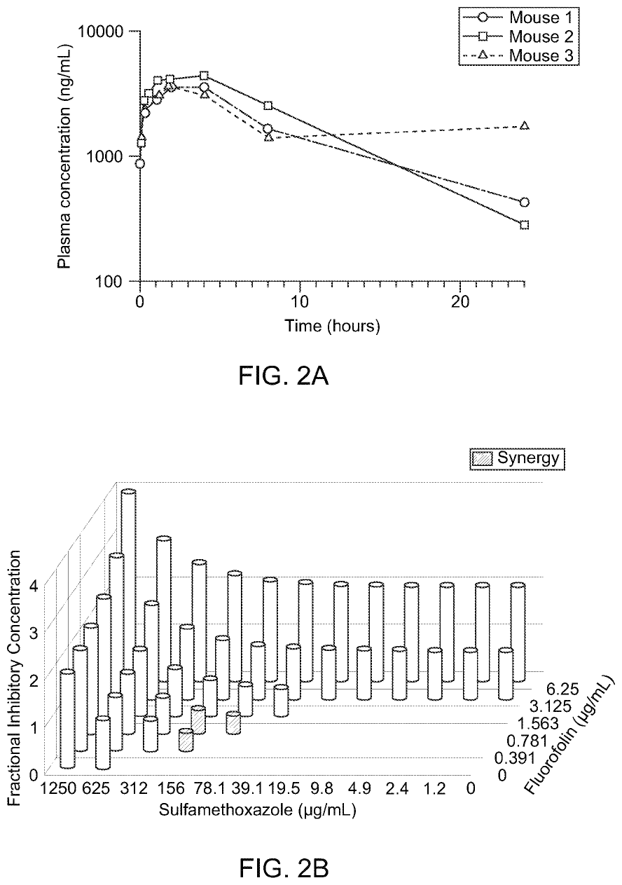

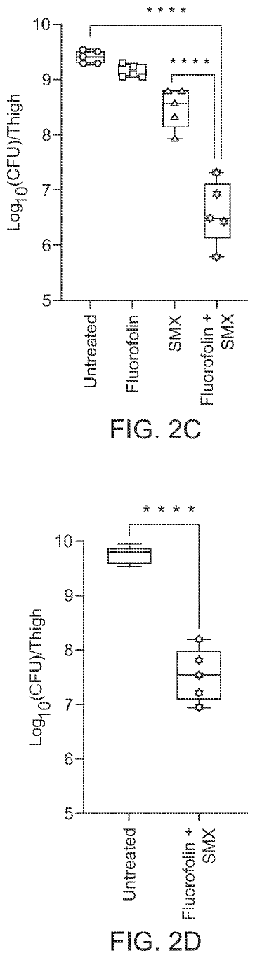

It was next sought to determine if fluorofolin has activity in an in vivo mouse infection model. In mice, fluorofolin displayed favorable plasma protein binding (71.7% bound, 91.9% recovery). Upon oral administration, fluorofolin achieved a peak concentration of 4.0 μg/mL with a half-life of 12.1 hours (FIG. 2A). Because the peak plasma concentration was so near the MIC for PA14 (3.1 μg/mL), we sought to further potentiate fluorofolin's antibiotic activity. It was found that the combination of fluorofolin and sulfamethoxazole (SMX) exhibited significant synergy in PA14 (FIG. 2B). Specifically, in the presence of well-tolerated doses of SMX (156 μg/mL), the MIC of fluorofolin was reduced to 0.4 μg/mL (FIG. 2B), establishing a promising therapeutic window with respect to concentration of fluorofolin achievable in vivo.

To investigate the ability of fluorofolin to clear a P. aeruginosa infection in vivo, a murine thigh infection model of PA14 was employed. The maximum tolerated dose of fluorofolin was determined to be 25 mg/kg administered subcutaneously (SC). As fluorofolin showed synergy with sulfamethoxazole, we co-administered fluorofolin with sulfamethoxazole (SMX) delivered at a dose of 100 mg/kg intraperitoneally. We note that this dose of SMX was previously used to mimic clinically-relevant levels of SMX used for cotreatment with TMP in humans. Mice were inoculated with 5.33 log CFU of P. aeruginosa PA14 CFU of PA14 and treated SC with fluorofolin at 25 mg/kg at 1 and 12 hours post infection. Thighs were harvested 24 hours post infection to determine bacterial load. The combination of fluorofolin (25 mg/kg) and SMX (100 mg/kg) significantly inhibited the growth of PA14 compared to both no treatment and SMX alone (P<0.0001, FIG. 2C).

It was also sought to support the feasibility of thymidine supplementation in vivo by testing the hypothesis that fluorofolin should remain active in the presence of thymidine supplementation. An additional mouse P. aeruginosa infection model was thus performed in which a group of mice fed with a thymidine-supplemented diet starting two days before infection was included. This group also showed a significant reduction in P. aeruginosa after 24 hours compared to untreated mice (FIG. 2D).

Fluorofolin Selectively Targets P. aeruginosa in the Presence of Exogenous Thymine

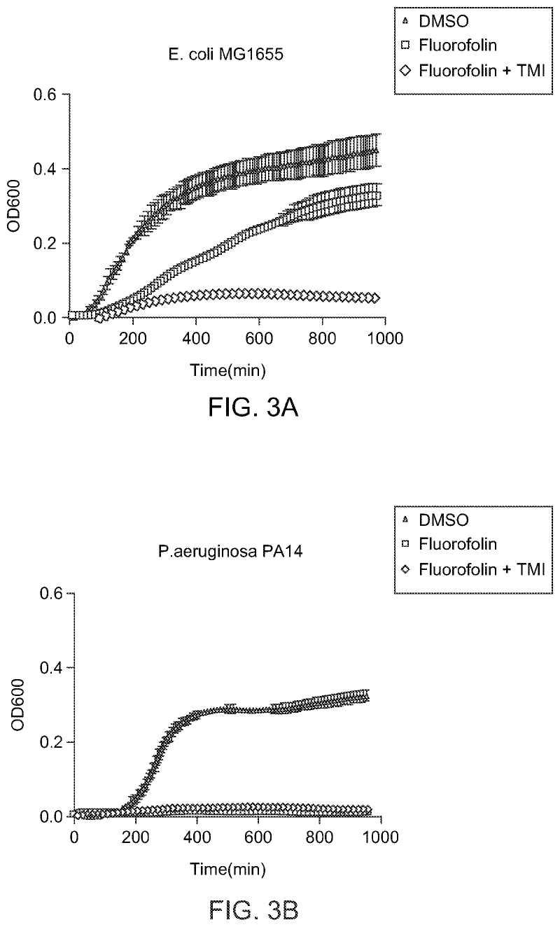

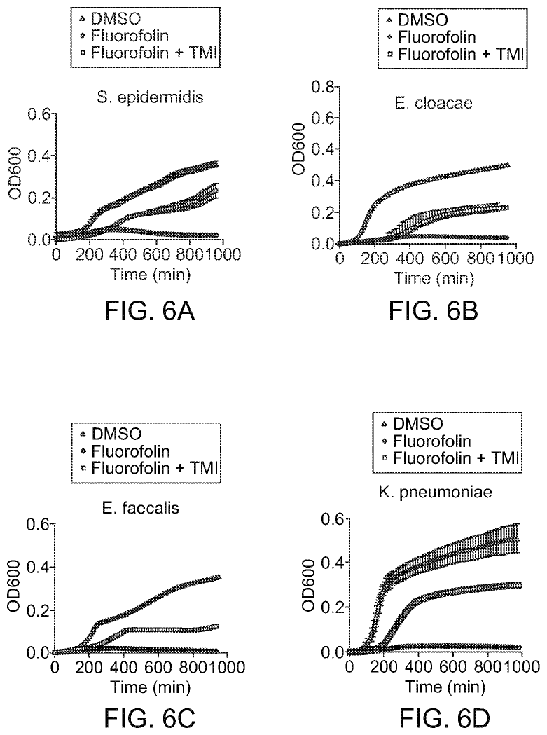

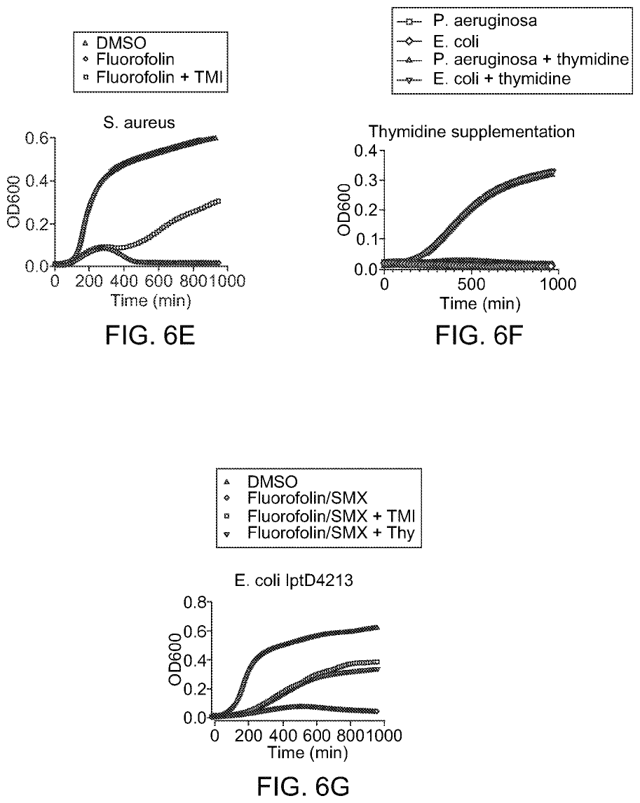

Supplementation of media with thymine, methionine, and inosine (TMI) can rescue DHFR-mediated growth inhibition by TMP in E. coli. It was confirmed that fluorofolin inhibition of E. coli MG1655 growth could also be rescued with TMI supplementation (FIG. 3A); however, TMI-supplementation was unable to rescue P. aeruginosa PA14 from fluorofolin treatment (FIG. 3B). These results are consistent with fluorofolin functioning as a broad-spectrum DHFR inhibitor and P. aeruginosa lacking the homologs for thymine kinase and thymidine phosphorylase necessary for utilizing exogenous thymine. It was thus hypothesized that by inhibiting DHFR in the presence of TMI supplementation, we could inhibit P. aeruginosa growth while allowing E. coli to grow. This hypothesis was directly tested by examining the effects of fluorofolin on co-cultures of E. coli MG1655 and P. aeruginosa PA14 in the presence or absence of TMI-supplementation. In the absence of TMI supplementation, fluorofolin effectively killed both E. coli and P. aeruginosa (FIG. 3C). But in the presence of TMI supplementation, fluorofolin selectively inhibited the growth of P. aeruginosa but not E. coli, as quantified by plating on selective media (FIG. 3C). In addition to the CFU counts, the selective killing of P. aeruginosa was evident in thymine-supplemented liquid cultures as P. aeruginosa cultures exhibit a characteristic blue-green hue from pyocyanin production that disappeared upon fluorofolin treatment. To demonstrate that the narrow-spectrum nature of fluorofolin is not specific to E. coli, we demonstrated that other species including S. epidermidis, E. cloacae, E. faecalis, K. pneumoniae, and S. aureus could also be rescued from fluorofolin growth inhibition by using TMI-supplementation (FIGS. 6A-6E). Rescue from fluorofolin inhibition was also shown in E. coli lptd34213 using thymidine supplementation alone (FIG. 6F), as well as in the presence of clinically-relevant concentrations of SMX (FIG. 6G). These results support the conclusion that P. aeruginosa PA14 is unusual in its inability to salvage exogenous thymine and demonstrate that thymine supplementation can convert fluorofolin from a broad-spectrum antibiotic to a narrow spectrum antibiotic.

### Fluorofolin Resistance Attenuates Virulence and is Rare in Clinical Isolates

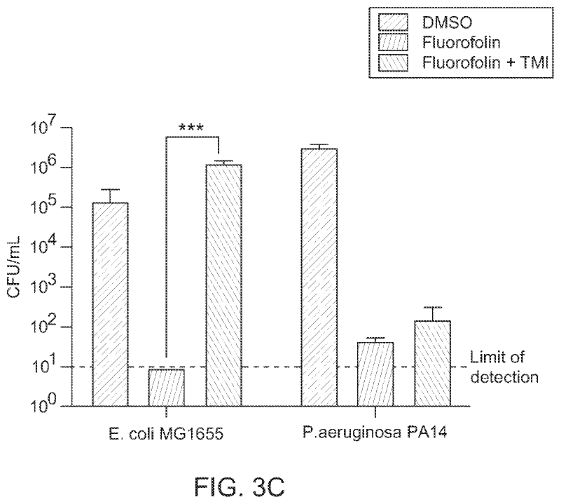

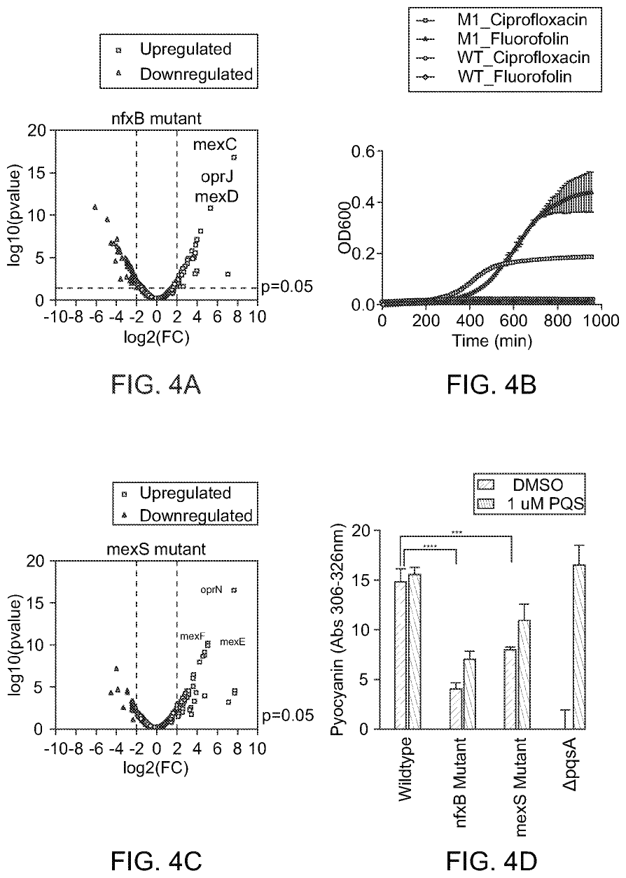

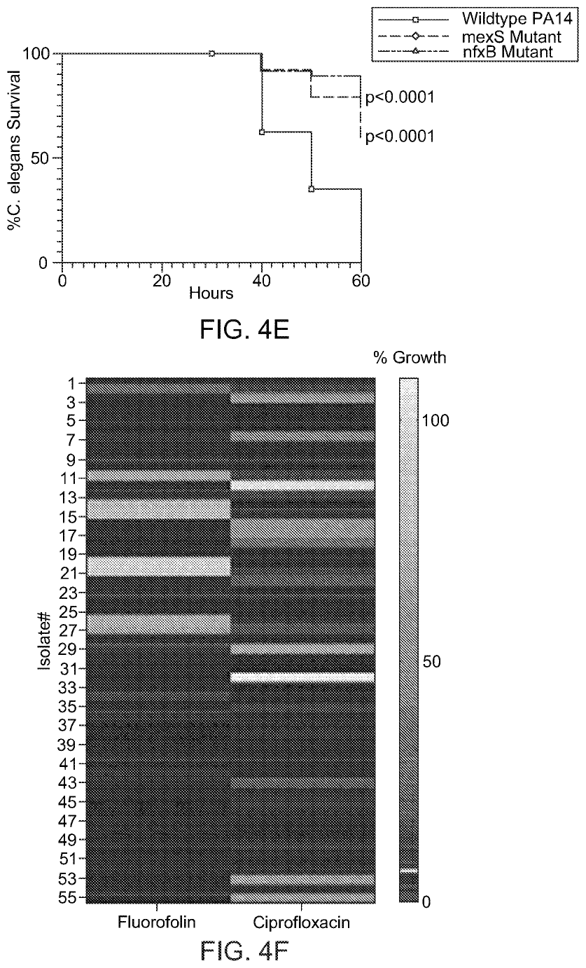

Because fluorofolin acts solely as a DHFR inhibitor, it was hypothesized that resistance to fluorofolin could more readily occur. We were indeed able to isolate two different fluorofolin-resistant mutants of P. aeruginosa PA14. One type of fluorofolin-resistant mutant was isolated through plating 10⁸ cells onto LB Agar plates containing 4×MIC fluorofolin. Resistance frequency on these plates was 1 in 1.5×10⁶ cells. While this mutation frequency is high, whole genome sequencing of these resistance mutants revealed that all the mutants mapped to a singular protein-coding gene, nfxB. Of the 8 mutants sequenced, 6 had a T39P point mutation, 1 had a L29R point mutation, and 1 had a premature stop codon at amino acid 115. The other class of fluorofolin-resistant mutant isolated arose through serial passaging of P. aeruginosa PA14 at 0.5×MIC fluorofolin for 10 passages. Whole genome sequencing revealed that the only protein-coding mutation in these mutants was a point mutation in mexS (L46F). MexS is an oxidoreductase that represses MexT, which in turn induces expression of an efflux pump, MexEF-OprJ. We confirmed that our mexS mutant caused upregulation MexEF-OprJ using RNA-seq (FIG. 4C).

NfxB is a transcriptional regulator protein that represses expression of the MexCD-OprN efflux pump. We confirmed that our nfxB mutants cause upregulation of MexCD-OprN using RNA-seq (FIG. 4A). P. aeruginosa nfXB mutants have also been shown to confer resistance to other antibiotics, including ciprofloxacin. As expected, the nfxB T39P mutants we isolated as resistant to fluorofolin were also cross-resistant to ciprofloxacin (FIG. 4B). Since the same mutations can confer resistance to fluorofolin and ciprofloxacin, we determined if the two antibiotics also have similarly high resistance frequency in our resistance plating assay. Indeed, resistance to ciprofloxacin (1 in 2.63×10⁶ cells) was similar to that of fluorofolin (1 in 1.5×10⁶ cells). These in vitro resistance frequencies are higher than expected of a clinically used antibiotic like ciprofloxacin, but below we demonstrate that they likely do not represent the resistance frequency observed in vivo.

Our animal infection study suggested that fluorofolin should be co-administered with SMX. The rate of resistance to fluorofolin/SMX combination treatment was therefore examined. To do so, 10⁸ CFU on plates containing 4×MIC of fluorofolin were plated and the lowest concentration of SMX at which we observed synergy (78.1 μg/mL). In these conditions we were unable to isolate resistance mutants, suggesting that the in vitro resistance frequency falls below our level of detection (<1 in 10⁸) in the context in which it would be used in vivo.

The physiological impacts of the nfxB and mexS efflux pump upregulation mutants were next explored. The nfxB and mexS mutants had significantly decreased production of the quorum sensing phenazine pyocyanin (P=0.0001 and P=0.0006 respectively) compared to wildtype PA14 (FIG. 4D). Efflux pump overexpression could increase secretion of quorum sensing precursors, thereby inhibiting the accumulation of the quorum sensing molecules themselves. We found that pyocyanin production could be partially rescued by addition of the quorum sensing molecule, PQS (FIG. 4E), which is known to both induce pyocyanin and have precursors that are susceptible to efflux. Quorum sensing and pyocyanin are known to affect P. aeruginosa virulence, suggesting that the fluorofolin resistant mutants could also affect P. aeruginosa pathogenesis. Both mutants were significantly less virulent than WT PA14 (P<0.0001) in a C. elegans N2 infection model (FIG. 4D). These results suggest that there is a trade-off in P. aeruginosa between fluorofolin resistance and virulence.

If there is a trade-off between fluorofolin resistance and virulence in vivo, we would predict that most clinical isolates of P. aeruginosa, which are virulent, should be susceptible to fluorofolin. Indeed, while efflux pump mutants are common in clinical isolates of many bacterial species, they are reported to be more rare in clinical isolates of P. aeruginosa, which could be explained by this fitness cost. To directly investigate the predominance of fluorofolin resistance in clinical P. aeruginosa isolates, we obtained the CDC & FDA Antibiotic Resistant P. aeruginosa Isolate Bank. We found that only 14.5% of the clinical isolates were resistant to fluorofolin at 50 μg/mL. This frequency compared favorably with the clinically-used antibiotic, ciprofloxacin, as 21.9% of the strains were resistant to ciprofloxacin at the same dose (FIG. 4F). While no folate inhibitors are in clinical use against P. aeruginosa, we found that all of the fluorofolin-resistant clinical isolates had a multidrug resistance cassette that contains an annotated dfrB5 integron with a mutant allele of DHFR known to confer clinical resistance to TMP in Klebsiella pneumoniae. This result confirms DHFR as the physiological target of fluorofolin in vivo and demonstrates that while DHFR mutations can result in fluorofolin resistance, such mutations are rare amongst existing P. aeruginosa clinical isolates. Finally, it was found that not all TMP-resistant DHFR mutations confer cross-resistance with fluorofolin, as strains 7, 18 and 23 of the CDC panel are fluorofolin-sensitive despite having dfrB2, another trimethoprim-resistant DHFR allele (FIG. 4F).

Importantly, while our nfxB efflux pump overexpression strain showed cross-resistance to both fluorofolin and ciprofloxacin (FIG. 4B), none of the P. aeruginosa isolates in our collection demonstrated cross-resistance to both antibiotics (FIG. 4F). This result supports the hypothesis that in P. aeruginosa, efflux pump upregulation readily confers antibiotic resistance in vitro, but that such mutations rarely accumulate in clinical settings due to their associated fitness cost. It was also encouraging to observe that P. aeruginosa clinical isolates expressing KPC-5 and NDM-1 carbapenemase genes, which are considered by the CDC to be critical targets of antibiotic research and development, remained sensitive to fluorofolin. Thus, while the rate at which fluorofolin resistance might emerge upon treatment in vivo remains to be determined, fluorofolin appears to be effective against most existing P. aeruginosa clinical isolates, including those of significant clinical interest.

### DISCUSSION

P. aeruginosa is a leading cause of nosocomial infections for which antibiotic development is urgently needed. P. aeruginosa infections are typically first treated with the fluroquinolone, ciprofloxacin. However, an increasing number of P. aeruginosa clinical isolates are reported to be resistant ciprofloxacin, diminishing clinical options. Furthermore, ciprofloxacin and other antibiotics currently used for P. aeruginosa (like piperacillin-tazobactam) are broad-spectrum, disrupting the host microbiome in a manner that often does not fully recover after treatment. There is consequently an urgent need for compounds that are both narrow-spectrum for P. aeruginosa and effective against existing multi-drug-resistant strains.

Here, new antibiotic compounds, including fluorofolin, capable of inhibiting the growth of P. aeruginosa through potent DHFR inhibition, Fluorofolin is effective both in vitro and in a mouse thigh infection model. Fluorofolin represents the first folate inhibitor that is effective at tolerated doses in P. aeruginosa. We thus used fluorofolin to demonstrate that P. aeruginosa, which lacks the ability to scavenge exogenous thymine present in most other bacterial species, can be selectively targeted by fluorofolin in the presence of thymine supplementation. While resistance to fluorofolin is possible, common mechanisms of resistance in vitro also confer a decrease in bacterial virulence in vivo, and thus most clinical isolates of P. aeruginosa are susceptible to fluorofolin.

Recent studies have highlighted the important of microbiome integrity for multiple aspects of human health including metabolic and immune system regulation. Broad-spectrum antibiotics can disrupt the microbiome leading to dysbiosis, highlighting the need for more targeted antibiotic approaches. Traditional efforts to develop narrow spectrum antibiotics have focused on targeting features that are specifically present in a bacterial species of interest. Here, however, we exploit the specific absence of a thymine salvage pathway in P. aeruginosa to show that a broad-spectrum inhibitor of folate synthesis can selectively inhibit the growth of this important pathogen in through thymine supplementation. Most human commensal bacteria have thymine kinase homologs and we confirmed that multiple bacterial species' sensitivity to fluorofolin can be rescued by thymine supplementation. We note that a few other species also lack thymine kinase including the human pathogens Helicobacter pylori and M. tuberculosis, suggesting that these pathogens could also be selectively targeted using a similar approach. Actinomycetes and their closely related genera Corynebacterium, Mycobacterium, and Rhodococcus have also been shown to lack thymidine kinase activity, but these bacteria represent a small subset of those present in the human microbiome and are predominantly found within skin communities. In humans, thymidine supplementation has been shown to be safe and is routinely used to reduce toxicity associated with methotrexate treatment.

Fluorofolin also lacks the ability to disrupt bacterial membranes. This divergence in activity improves the therapeutic index of fluorofolin, likely due to minimizing off-target effects on mammalian membranes. However, this loss in mechanism of action also allows for resistance against fluorofolin to develop more easily in vitro. We were able to isolate two fluorofolin resistant mutants in vitro, which were both attributed to the overexpression of efflux pumps (MexCD-OprJ in one mutant and MexEF-OprN in the other). MexCD-OprJ overexpression has been shown to confer resistance to cefpirome and quinolones⁶, while MexEF-OprN overexpression has been shown to confer resistance to imipenem, chloramphenicol, and quinolones. Interestingly, while these mutants are isolated in lab settings, they are rarely isolated from P. aeruginosa clinical samples. Here we find that fluorofolin-resistant mutants that overexpress these efflux pumps have significantly reduced virulence, which would explain their low frequency in pathogenic isolates. These findings are consistent with studies from P. aeruginosa PAO1 demonstrating that overexpression of efflux pumps decreases type III secretion, secretion of virulence factors, and swarming. MexCD-OprJ has been suggested to efflux 2-heptyl-4-quinolone (HHQ) while MexEF-OprN has been suggested to efflux kynurenine⁴⁴. HHQ and kynurenine are precursors of Pseudomonas quinolone signal (PQS), a key molecule in regulating Pseudomonas quorum sensing and virulence. Together, these data suggest that high efflux pump levels secrete quorum sensing precursors, preventing the synthesis of quorum sensing molecules that promote virulence, and thereby reducing virulence. In support of this hypothesis, it was demonstrated that addition of exogenous PQS was able to partially restore pyocyanin production to the fluorofolin resistant mutants. Co-treatments with other antibiotics are an additional approach to reducing resistance emergence. It was demonstrated that co-treatment with SMX reduced fluorofolin resistance to undetectable levels in vitro. Meanwhile, mutants in MexCD-OprJ are hypersusceptible to imipenem while mutants in MexEF-OprN are hypersusceptible to aminoglycoside and β-lactams, such that combination therapies of fluorofolin with these antibiotics may also prove effective at addressing any residual resistance to fluorofolin.

As will be understood by those familiar with the art, the present invention may be embodied in other specific forms without departing from the spirit or essential characteristics thereof. Accordingly, the disclosures and descriptions herein are intended to be illustrative, but not limiting, of the scope of the invention which is set forth in the following claims.

### Materials and Methods

### Fluorofolin Synthesis

Synthesis of Flurofolin was made by the general scheme shown below.

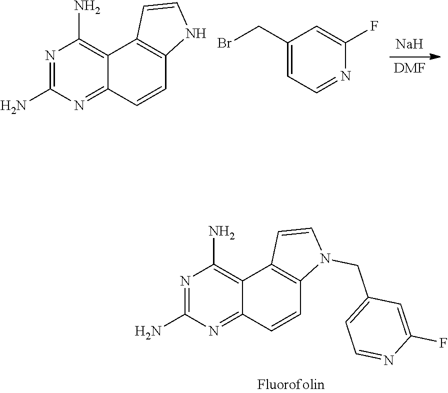

To a stirred solution of 7H-Pyrrolo[3, 2-f]quinazoline-1,3-diamine (1.0 mmol) in dry DMF (20 mL) was added NaH (1.2 mmol). The resulting reaction mixture was stirred at 0° C. for 0.5 h. Then corresponding bromide (1.5 mmol) was added. The reaction mixture was stirred at 0° C. for 1 h. The solvent was removed under reduced pressure and the residue was purified by column chromatography on silica gel, eluting with 10:1 DCM:MeOH containing 1% Et₃N to give the desired compound as a solid.

¹H NMR (400 MHz, DMSO-d₆) δ 8.16 (d, J=5.2 Hz, 1H), 7.71 (d, J=9.0 Hz, 1H), 7.66 (d, J=3.2 Hz, 1H), 7.18 (d, J=3.2 Hz, 1H), 7.05 (d, J=9.0 Hz, 1H), 7.00-6.98 (m, 1H), 6.84 (d, J=1.5 Hz, 1H), 6.79 (s, 2H), 5.80 (s, 2H), 5.64 (s, 2H).

MS (ESI): [M+H⁺]309.36

### Experimental Models and Subject Details

Bacterial strains and growth conditions Bacterial strain information is provided in Table 1. Where listed, growth media were prepared according manufacturer recommendations: LB Broth and LB Broth supplemented with 0.3 mM thymine (BD Biosciences 244610, Alfa Aesar A15879), 0.3 mM methionine (Sigma-Aldrich M9625), 0.3 mM inosine (EMD Millipore 4060), Mueller Hinton II Broth (Cation-Adjusted)/(CAMHB) (BD 212322) or 0.3 mM thymidine (Sigma-Aldrich T1895), Gutnick Minimal Media (1.0 g/L K₂SO₄, 13.5 g/L K₂HPO₄, 4.7 g/L KH₂PO₄, 0.1 g/L of MgSO₄-7H₂O, 10 mM NH₄Cl as a nitrogen source, and 0.4% w/v glucose as a carbon source)⁶².

### Animal Models

Pharmacokinetics determination: Care and handling of male CD-1 mice approximately 6-8 weeks old conformed to institutional animal care and use policies as carried out at Pharmaron, Inc. (Beijing, ROC).

P. aeruginosa thigh infection model and MTD studies: Care and handling of Female 5-6-week-old CD-1 conformed to institutional animal care and use policies as carried out at University of North Texas Health Science Center (Fort Worth, Texas). Rodents were fed either base chow or base diet supplemented with 1.8 g/kg thymidine (Sigma T9250) obtained from Envigo starting two days before infection.

### MIC Results

The minimum inhibitory concentration is defined as the lowest concentration of antibiotic at which no visible growth was detected after 16 hours at 37° C. Overnight cultures were diluted 1:150 in LB Broth and added to a 96-well plate. Antibiotics were serially diluted 1:2 and added to columns of the 96-well plate and grown at 37° C. with continuous shaking. Cell growth was measured by optical density (OD₆₀₀). MIC assays were performed in either BioTek Synergy HT (Winooski, VT) or Tecan InfiniteM200 Pro (Männedorf, CH) microplate readers.

For MICs calculations performed by WuXi (Shanghai, China), MIC was calculated as the lowest concentration that inhibits visible growth after 18 hours. 4-8 bacterial colonies of strains of interest were vortexed in saline and adjusted to an OD₆₀₀ of 0.2. Strains were diluted 1:200 into CAMHB media into 96-well plates. Antibiotics were serially diluted 1:3 in DMSO, and 1 μL of each dilution was added to bacteria. The plates were incubated for 18-20 hours at 37° C. before observation.

### Colony Forming Units (CFU) Counting

P. aeruginosa PA14 overnight cultures were diluted 1:100 and grown to exponential phase (OD₆₀₀=0.4-0.6). Cultures were diluted 1:10 and treated with antibiotic. At each time point, 150 μL of culture was removed, serial diluted 1:10 six times, and plated onto LB Agar plates. Plates were grown overnight at 37° C. after which visible colonies were counted. CFU/mL was reported from dilutions in which ˜10-100 single colonies were visible.

### Membrane Potential and Permeability Assay

Overnight P. aeruginosa PA14 or E. coli lptD4213 cultures were diluted 1:100 and grown to mid exponential phase at 37° C. Cultures were diluted 1:10 into PBS and treated with antibiotics for 15 minutes. P. aeruginosa PA14 was stained with TO-PRO-3 (640 nm excitation, 670/30 nm emission) to measure cell membrane integrity. E. coli lptD4213 was stained with both TO-PRO-3 and DiOC2(3) (ThermoFisher B34950) to measure cell membrane integrity and membrane potential. DiOC2(3) was evaluated as a ratio of green (488 nm excitation, 525/50 nm emission) to red (488 nm excitation, 610/20 nm emission) (Novo et al., 1999). The LSRII flow cytometer (BD Biosciences) at the Flow Cytometry Resource Facility, Princeton University, was used to measure the fluorescent intensities of both dyes in response to antibiotic treatment. 100,000 events were recorded for each data file. Gates for permeabilization were determined using Polymixin B (Sigma-Aldrich P1004) and untreated controls. Gates for depolarization were determined using CCCP as a positive control. Data was analyzed using FlowJo v10 software (FlowJo LLC, Ashland, OR).

### MexAB-OprM Transposon Mutants

P. aeruginosa PA14 transposon mutants were generated by the Ausubel Lab (http://ausubellab.mgh.harvard.edu/cgi-bin/pa14/home.cgi). The MICs of fluorofolin and TMP against strains with disrupted MexA, MexB, and OprM were determined as above and compared to the parental strain. As transposon mutants in MexB were represented twice in this collection, the MIC was confirmed across both mutant strains.

### Hemolysis

Defibrinated sheep red blood cells (Lampire 50414518) were diluted to 6×10⁶ cells/mL, pelleted, and washed 3× with PBS. Samples were treated at 37° C. with shaking for 1 hour and then centrifuged. Supernatants were collected and absorbances were measured at 405 nm in a Tecan InfiniteM200 Pro (Männedorf, CH) microplate reader. Percentage hemolysis was calculated compared to 100% lysis by Triton X-100 (1% v/v) (Sigma-Aldrich X100RS).

### Mammalian Cell Cytotoxicity

HK-2 (500 cells/well) (ATCC CRL-2190), HLF (500 cells/well) (Cell Applications 506K-05a), WI-38 (500 cells/well) (ATCC CCL-75) or PBMC (5000 cells/well) (TPCS PB010C) cells were seeded in white, opaque, 384-well plates. After 24 hours, DMSO or compounds were added in 3-fold dilutions and incubated for 72 hours. For PBMC, CellTiter-Glo Reagent was added in equal volume and incubated for 30 min after which luminescence was read. For other cell types, CyQUANT Detection Reagent was added in equal volume and incubated for 1 hour after which fluorescence was read with standard green filter set (508/527 nm ex). Cell toxicity was evaluated by Pharmaron, Inc. (Beijing, ROC).

### Metabolomics

Overnight P. aeruginosa PA14 cultures were diluted 1:150 in Gutnick Minimal Media and grown to early-mid exponential phase (OD₆₀₀=0.4-0.6). Cultures were treated with either 6.3 μg/mL fluorofolin (2×MIC) or 250 ug/mL Trimethoprim (2×MIC) (Chem-Impex 01634) for 15 min. Metabolites were extracted by vacuum filtering 15 mL of treated cells using 0.45 μm HNWP Millipore nylon membranes and placing the filters into an ice-cold quenching solution 40:40:20 Methanol:Acetonitrile:H2O. Extracts were kept on dry ice for 1 hour and centrifuged at 16,000 g for 1 hour at 4° C. The supernatant was kept at −80° C. until mass spectrometry analysis.

LC-MS analysis of metabolites was performed on Orbitrap Exploris 240 mass spectrometer coupled with hydrophilic interaction liquid chromatography (HILIC)⁶⁴. HILIC was on an XBridge BEH Amide column (2.1 mm×150 mm, 2.5 μM particle size; Waters, 196006724), with a gradient of Solvent A (95 vol % H2O 5 vol % acetonitrile, with 20 mM ammonium acetate and 20 mM ammonium hydroxide, pH 9.4), and solvent B (acetonitrile). Flow rate was 0.15 mL/min, and column temperature was set at 25° C. The LC gradient was: 0-2 min, 90% B; 3-7 min, 75% B; 8-9 min, 70% B; 10-12 min, 50% B; 12-14 min, 25% B; 16-20.5 min, 0.5% B; 21-25 min, 90%. The orbitrap resolution was 180,000 at m/z of 200. The maximum injection time was 200 ms, and the automatic gain control (AGC) target was 1000%. Raw mass spectrometry data were converted to mzXML format by MSConvert (ProteoWizard). Pick-peaking was done on El Maven (v0.8.0, Elucidata).

In Vitro DHFR E. coli

As previously in, purified E. coli dihydrofolate reductase (FolA) was purified by Genscript (Piscataway, NJ). Enzyme activity was measured on a QuantaMaster 40 Spectrophotometer (Photon Technology International Inc., Edison, NJ) using the DHFR reductase assay kit with slight modifications. E. coli FolA was diluted 1:1000 into 1× assay buffer. 100 μL of this mixture with or without compound was added to BRAND® UV cuvette (Sigma-Aldrich, BR759200) and sample transmitted light intensity at 340 nm was measured for 100 s at 1 kHz sampling. Readings were averaged for every 1 Hz and the activity of each sample was calculated from the slope(s) of a linear regression of the log transformed intensity measurements on MATLAB. To account for enzyme stability, measurements were normalized to a standard condition (60 M NADPH and 100 M DHF) measured immediately before the sample of interest. The relative activity was calculated as (βsample−βnoEnzyme)/(βstandard−βnoEnzyme).

### Human DHFR In Vitro Assay

Human purified DHFR was purchased from R&D Systems (8456-DR). DHFR activity was assayed by monitoring the decrease in absorbance by NADPH at 340 nM. DHFR enzyme (0.5 μg/mL), dihydrofolic acid (100 μM), and different concentrations of methotrexate, fluorofolin, or DMSO control was dissolved in 200 μL of Tris buffer (pH 7.5, Tris salt concentration 25 mM). Reaction was initiated by adding NADPH in a 10× stock (1 mM for final concentration 100 μM), and absorbance at 340 nM was monitored over time by Cytation 5 reader (Agilent). Activity was normalized to the DMSO control.

### Molecular Docking

As the structure of P. aeruginosa DHFR has not been solved, we used AlphaFold^{30,31} to derive the enzyme's three-dimensional structure from its sequence (UniProt ID: 6XG5)³². Following the acquisition of the protein structure, we introduced the coenzyme NADPH to the structure, aligning it on the experimentally characterized human DHFR in a complex with IRS-17, providing a structural reference for subsequent steps.

The structures of fluorofolin and trimethoprim were translated from SMILES representation using the RDKit chemoinformatic package. After preparing the enzyme, coenzyme, and ligand structures, we defined a cubic grid box of dimensions 20×20×20 Angstroms centered around the active site of the reference human DHFR-IRS-17 complex. This box serves as the search space for potential binding sites in our docking simulations. We executed the docking simulation using the AutoDock Vina^{33,34} forcefield, with an exhaustiveness parameter set to 64 to ensure comprehensive sampling of the search space.

### Checkerboard Assay

Cells were seeded in a similar manner as described above for MIC calculations. Sulfamethoxazole (Chem-Impex 00821) was diluted 1:2 down the rows of the plate, while fluorofolin was diluted 1:2 down the column of the plate. Fractional inhibitory concentrations (FICs) were calculated as [fluorofolin]/MIC_{Fluorofolin}+[SMX]/MIC_{SMX} where [fluorofolin] and [SMX] are the concentrations of compounds in given well, which are dividing by the concentration of drug at the MIC for each compound. FIC values less than or equal to 0.5 are considered synergistic.

### Growth Competition Assay

Overnight cultures of P. aeruginosa PA14 or E. coli MG1655 were diluted 1:150 into LB Broth in the presence of DMSO or 50 μg/mL fluorofolin with or without TMI supplementation and grown for 16 hours at 37° C. Cultures of each species were grown separately as well as being mixed 1:1. Cultures were plated onto LB Agar or Pseudomonas Selection Agar (Sigma-Aldrich 17208) and CFU counting was carried out as described above. To control for appropriate, Pseudomonas selection, E. coli MG1655 was plated onto Pseudomonas Selection Agar and an absence of colonies was observed. The number of colonies on Pseudomonas Selection Agar plates was reported as the CFU/mL of P. aeruginosa. To calculate CFU/mL of E. coli MG1655, CFU/mL were determined from LB Agar plates, and the CFU/mL of P. aeruginosa were subtracted from these values.

### Drug Accumulation Assay

Overnight PA14 cultures were back-diluted to early-mid exponential phase (OD600=0.4-0.6). The assay was initiated with treatment of the culture with either 5.0 μM fluorofolin or 5.0 μM trimethoprim. A DMSO treated culture was utilized as a control. At time points of 30, 60, and 90 min, a 10 mL aliquot was collected out of the 120 mL parent culture (in triplicate) and pelleted by centrifugation at 3,500 rpm at 4° C. The supernatant was then removed, and the pellet was washed with ice cold 0.85% NaCl solution. Following suspension of the cell pellet in 1 mL of 2:2:1 CH₃CN:MeOH:H₂O, samples were subjected to four cycles of freeze-thaw cell lysis using dry ice in 95% ethanol/ice water. Prior to each freeze phase, samples were vortexed for 10 s to ensure adequate mixing. Samples were subsequently pelleted at 15,000 rpm for 5 min with the supernatant being subjected to filtration using a 0.22 μm SpinX centrifuge tube filter. The resulting cell lysate samples were analyzed utilizing verapamil as an internal standard. For LC/MS analysis, sample components were separated using a Chromolith SpeedRod column, using a gradient of 10-100% CH₃CN/H₂O acidified with 0.1% v/v formic acid, with an Agilent 1260 Liquid Chromatography coupled to an Agilent 6120 quadruple mass spectrometer.

Compound accumulation was realized using the selective ion monitoring (SIM) mode to quantify peak integration for a compound and the internal standard using their respective m/z values. Compound peaks were confirmed using a scanning mode that detected the compound peak using an m/z range of 100-1000. Peak area integration values were determined and a ratio of the peak area for the compound to the peak area for the verapamil internal standard was calculated and compound concentration was then determined from the compound calibration curve. The calculated concentration of the compound in each sample was then normalized using the bacterial culture OD₆₀₀ value. Compound accumulation versus time plots were generated using GraphPad Prism Version 9.4.1. Compound accumulation area under the curve (calculated in Microsoft Excel Version 16.65) was determined for each bacterial strain-compound combinations and these were compared via statistical analysis (unpaired t-test) GraphPad Prism Version 9.4.1.

### Fluorofolin Resistance Screens

For resistance passaging, P. aeruginosa PA14 was grown overnight at 37° C. in a 96-well plate similarly to MIC assays in duplicate. The wells corresponding to 0.5×MIC was selected and struck out on LB Agar plates in the absence of antibiotic to select for stable resistance. Single colonies were picked, and inoculated into fresh LB Broth. This process was repeated for a total of ten passages. At each passage, the MIC was recalculated and compared to a culture that had not been previously exposed to fluorofolin was also grown as a control to confirm antibiotic potency. Cells from each passage were stored as a frozen stock.

To isolate resistant mutants on a plate, 10⁸ CFU of P. aeruginosa PA14 was plated on LB Agar plates containing 4×MIC fluorofolin, 4×MIC ciprofloxacin, or 4×MIC fluorofolin with 4× the minimal concentration of SMX at which synergy was observed. Plates were grown at 37° C. for 48 hours after which individual colonies were picked and restruck onto fresh 4×MIC fluorofolin and grown in 4×MIC fluorofolin in LB Broth to confirm resistance. Resistant mutants were maintained in a frozen stock.

To confirm the identity of resistance mutations, whole genome sequencing was performed and compared to the parental strain of PA14. Briefly, genomic DNA was isolated from a strain of interest using the DNAeasy Blood and Tissue Kit (Qiagen 69504). Once DNA was extracted and its quality was confirmed, the DNA was sequencing using an Illumina NextSeq 2000. Sequencing and variant calling was performed at Seq Center (Pittsburgh PA).

### RNA Sequencing

RNA was extracted from overnight cultures of wildtype PA14 or mutant strains. Cultures were pelleted at 4° C., resuspended in Trizol (Ambion 10296010), and incubated at 65° C. before addition of chloroform. The aqueous layer was collected and RNA was isolated using a mirVana RNA extraction kit (ThermoFisher AM1560). DNAse treatment was performed on RNA using recombinant DNase I (Sigma-Aldrich 04716728001). RNA samples were sent to Seq Center (Pittsburgh, PA) for sequencing using the Illumina Stranded RNA library preparation with RiboZero Plus rRNA depletion.

### Pharmacokinetic Analysis

Pharmacokinetic properties were determined after a single dose of 200 mg/kg fluorofolin given PO. Plasma samples were taken from three mice at times 0.083, 0.25, 0.5, 1, 2, 4, 8, and 24 hours and quantitative analysis was performed using LC/MS/MS. Half-life was determined from plasma concentration after fluorofolin levels reached pseudo-equilibrium (4 hours for mouse 1 and 2, and 2 hours for mouse 3). Pharmacokinetic values were estimated using a non-compartmental model generated from WinNonlin 6.1. Pharmacokinetic analysis was carried out by Pharmaron, Inc. (Beijing, ROC).

### Serum Binding

1 μM of fluorofolin was added to a mouse plasma solution or to a buffer only control. An initial t=0 sample was collected. fluorofolin was incubated with plasma for 6 hours after which dialysis was performed. After dialysis, supernatant was collected, and the amount of fluorofolin was determined by LC-MS/MS to determine the % Unbound of fluorofolin. Serum binding parameters were determined as follows:
1. % Bound=100%−% Unbound
2. Log K=Log (% Bound/100−% Bound)
3. % Remaining=Area-ratio₆ₕᵣ/Area-ratio₀ₕᵣ×100%
4. % Recovery was determined as (Area-ratio_{buffer-chamber}+Area-ratio_{plasma-chamber})/(Area-ratio_{Total sample})×100
   Serum binding analysis was performed by Pharmaron, Inc. (Beijing, ROC).
   C. elegans Maintenance and Toxicity Assay

C. elegans N2 worms were maintained on E. coli OP50-coated Nematode Growth Medium (NGM) plates prior to experiments. For P. aeruginosa-coated plates, overnight cultures were diluted to OD₆₀₀=1, spread onto NGM plates, incubated overnight at 37° C., and equilibrated to 25° C. To synchronize worms for virulence assays, young adult hermaphrodites were bleached to obtain eggs and synchronized L4 worms were collected 2 days post-bleaching. For virulence assays, synchronized L4 worms were transferred to P. aeruginosa plates. Worms were counted at time t=0, 30, 40, 50 and 60 hours to assess viability. Worms were declared dead if they lacked movement after gently poking with forceps. P values were calculated using a Mantel cox test to compare mutant virulence to wildtype PA14 (Prism 9).

### CDC Clinical Isolate Panel

Clinical isolates⁴⁵ were inoculated into LB Broth a 96-well flat bottom plate and grown overnight to stationary phase at 37° C. The follow day, strains were diluted 1:150 into fresh LB Broth with fluorofolin or ciprofloxacin at 50 μg/mL or vehicle control wells and incubated at 37° C. overnight. Percent growth was calculated by dividing OD₆₀₀ of fluorofolin antibiotic treated wells to vehicle only wells.

### Pyocyanin Production

Overnight cultures of PA14, PA14 ΔpqsA⁶⁵ and each mutant were grown in biological triplicate and the OD₆₀₀ of the cultures were measured. Cell-free supernatants were calculated through centrifugation, and 100 uL were added to a 96-well plate in duplicate. The integrated absorbance spectrum from 306-326 nm was taken in a Tecan InfiniteM200 Pro (Männedorf, CH) to determine pyocyanin levels in each sample. A ΔpqsA sample was used to subtract out any background values and pyocyanin levels were normalized by the ratio of OD₆₀₀ between wildtype PA14 and the mutants to account for any slight differences in growth.

### Maximum Tolerated Dose (MTD)

MTD was determined through administration of compounds at increasing dosage until the maximum dose before adverse reactions were observed. Doses were increased in a stepwise manner from 1 mg/kg to 5, 10, 25, 50 mg/kg. Mice were observed for the adverse effects including respiration, piloerection, startle response, skin color, injection site reactions, hunched posture, ataxia, salivation, lacrimation, diarrhea, convulsion, and death. MTD was evaluated by the University of North Texas Health Science Center (Fort Worth, Texas).

### In Vivo PA14 Infection

Female 5-6-week-old CD-1 mice were made neutropenic through intraperitoneal (IP) cyclophosphamide treatment (Cytoxan) prior to this study. On day 0, mice were infected intramuscularly (IM) with 1×10⁶ CFU/thigh PA14. Mice were treated subcutaneously (SC) with fluorofolin 1- and 12-hours post-infection. For cotreatment, mice were treated with SMX IP at 1- and 12-hours post infection. Mice were euthanized by CO₂ after which thighs were removed and placed into sterile PBS, homogenized, and serially diluted onto BHI and charcoal plates for CFU counting. In vivo efficacy was evaluated by the University of North Texas Health Science Center (Fort Worth, Texas).

### Statistical Information

For all assays showing error bars, we use the arithmetic mean and standard deviation across multiple biological replicates as our measures of center and spread. The number of replicates for each experiment type and the type of statistical test used to determine significance are included in respective figure legends.

1. A composition comprising:
a dihydrofolate reductase inhibitor of Formula I and/or a salt thereof: 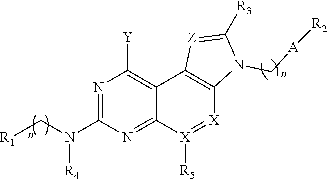
wherein R₁, R₃, R₄ and R₅ are independently selected from the group consisting of hydrogen, alkyl, alkenyl, heteroalkyl, cycloalkyl, heterocycloalkyl, aryl, heteroaryl, amide, halo, and urea, wherein the alkyl, alkenyl, heteroalkyl, cycloalkyl, heterocycloalkyl, aryl, heteroaryl, and amide are optionally substituted with one or more substituents selected from the group consisting of (C₁-C₁₀)-alkyl, (C₁-C₁₀)-alkenyl, cycloalkyl, heterocycloalkyl, aryl, heteroaryl, alkoxy, amide, sulfonamide, urea, halo, cyano, hydroxy, C(O)OR₆, and C(O)R₇, wherein R₆ is selected from the group consisting of hydrogen, alkyl and alkenyl and R₇ is selected from the group consisting of hydrogen, alkyl, alkenyl, heteroalkyl, cycloalkyl, heterocycloalkyl, aryl, heteroaryl and NR₈R₉, wherein R₈ and R₉ are independently selected from the group consisting of hydrogen, alkyl, alkenyl, aryl and heteroaryl; and
wherein R₂ is selected from the group consisting of halo, fluoroalkyl, and cyano; and
wherein A is heteroarylene; and
wherein X and Z are independently selected from the group consisting of C, N, O and S; and
wherein Y is selected from the group consisting of hydrogen, OH, alkoxy, and NR₁₀R₁₁, wherein R₁₀ and R₁₁ are independently selected from the group consisting of hydrogen, alkyl, heteroalkyl, cycloalkyl, heterocycloalkyl, alkenyl, aryl, heteroaryl, amide, sulfonamide, urea, alkylene-aryl, alkylene-heteroaryl, and C(O)R₁₂ wherein R₁₂ is selected from the group consisting of hydrogen, alkyl, alkenyl, heteroalkyl, cycloalkyl, heterocycloalkyl, aryl and heteroaryl and wherein R₁₀ and R₁₁ may optionally form a ring structure, wherein the aryl, heteroaryl, alkylene-aryl and alkylene heteroaryl are optionally substituted with one or more substituents selected from the group consisting of alkyl, alkenyl, alkynyl, halo, and alkynylene-alkylsilane; and n is an integer from 0 to 5; and
and a potentiator of the dihydrofolate reductase inhibitor of Formula I and/or salt thereof.

2. The composition of claim 1 having a minimum inhibitory concentration (MIC) against P. aeruginosa of 0.1 μg/mL to 1 μg/mL.

3. The composition of claim 1, wherein the potentiator is present in an amount of 50 μg/mL to 300 μg/mL.

4. The composition of claim 1, wherein A is pyridinylene.

5. The composition of claim 4, wherein R₂ is selected from the group consisting of halo and fluoroalkyl.

6. The composition of claim 5, wherein the dihydrofolate reductase inhibitor of Formula I and/or a salt thereof is of the formula:

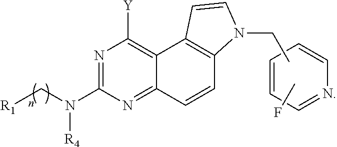

7. The composition of claim 1, wherein A is thiophenylene.

8. The composition of claim 4, wherein R₂ is cyano.

9. The composition of claim 8, wherein the dihydrofolate reductase inhibitor of Formula I and/or a salt thereof is of the formula:

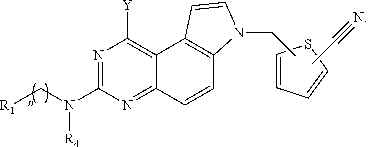

10. The composition of claim 1, wherein the potentiator comprises sulfamethoxazole.

11. A composition comprising:
a dihydrofolate reductase inhibitor of Formula I and/or a salt thereof: 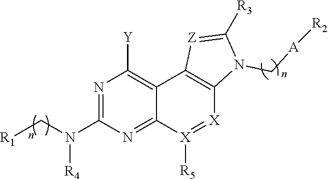
wherein R₁, R₃, R₄ and R₅ are independently selected from the group consisting of hydrogen, alkyl, alkenyl, heteroalkyl, cycloalkyl, heterocycloalkyl, aryl, heteroaryl, amide, halo, and urea, wherein the alkyl, alkenyl, heteroalkyl, cycloalkyl, heterocycloalkyl, aryl, heteroaryl, and amide are optionally substituted with one or more substituents selected from the group consisting of (C₁-C₁₀)-alkyl, (C₁-C₁₀)-alkenyl, cycloalkyl, heterocycloalkyl, aryl, heteroaryl, alkoxy, amide, sulfonamide, urea, halo, cyano, hydroxy, C(O)OR₆, and C(O)R₇, wherein R₆ is selected from the group consisting of hydrogen, alkyl and alkenyl and R₇ is selected from the group consisting of hydrogen, alkyl, alkenyl, heteroalkyl, cycloalkyl, heterocycloalkyl, aryl, heteroaryl and NR₈R₉, wherein R₈ and R₉ are independently selected from the group consisting of hydrogen, alkyl, alkenyl, aryl and heteroaryl; and
wherein R₂ is selected from the group consisting of halo, fluoroalkyl, and cyano; and
wherein A is heteroarylene; and
wherein X and Z are independently selected from the group consisting of C, N, O and S; and
wherein Y is selected from the group consisting of hydrogen, OH, alkoxy, and NR₁₀R₁₁, wherein R₁₀ and R₁₁ are independently selected from the group consisting of hydrogen, alkyl, heteroalkyl, cycloalkyl, heterocycloalkyl, alkenyl, aryl, heteroaryl, amide, sulfonamide, urea, alkylene-aryl, alkylene-heteroaryl, and C(O)R₁₂ wherein R₁₂ is selected from the group consisting of hydrogen, alkyl, alkenyl, heteroalkyl, cycloalkyl, heterocycloalkyl, aryl and heteroaryl and wherein R₁₀ and R₁₁ may optionally form a ring structure, wherein the aryl, heteroaryl, alkylene-aryl and alkylene heteroaryl are optionally substituted with one or more substituents selected from the group consisting of alkyl, alkenyl, alkynyl, halo, and alkynylene-alkylsilane; and n is an integer from 0 to 5; and
a thymine component.

12. The composition of claim 11, wherein the thymine component comprises thymine, methionine, and/or inosine (TMI).

13. The composition of claim 11, wherein the thymine component is employed in an amount of 0.5 g/kg to 5 g/kg.

14. The composition of claim 11 further comprising a potentiator of the dihydrofolate reductase inhibitor of Formula I and/or salt thereof.

15. The composition of claim 14, wherein the potentiator is present in an amount of 50 μg/mL to 300 μg/mL.

16. The composition of claim 11, wherein A is pyridinylene.

17. The composition of claim 16, wherein R₂ is selected from the group consisting of halo and fluoroalkyl.

18. The composition of claim 17, wherein dihydrofolate reductase inhibitor of Formula I and/or a salt thereof is of the formula:

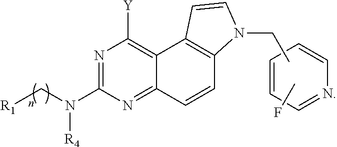

19. The composition of claim 1, wherein A is thiophenylene.

20. The composition of claim 19, wherein R₂ is cyano.

21. The composition of claim 20, wherein the dihydrofolate reductase inhibitor of Formula I and/or a salt thereof is of the formula:

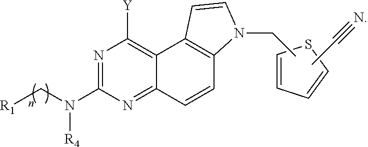

22. The composition of claim 14, wherein the potentiator comprises sulfamethoxazole.

23. The composition of claim 11, wherein the composition selectively kills or inhibits growth of bacteria incapable of utilizing exogenous thymine.

24. The composition of claim 23, wherein the bacteria comprises P. aeruginosa.

25. A method comprising:
treating an infection of pathogenic bacteria by administering to a patient in need thereof a therapeutically effective amount of a composition comprising a dihydrofolate reductase inhibitor of Formula I and/or a salt thereof: 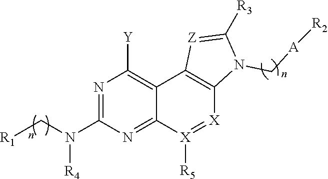
wherein R₁, R₃, R₄ and R₅ are independently selected from the group consisting of hydrogen, alkyl, alkenyl, heteroalkyl, cycloalkyl, heterocycloalkyl, aryl, heteroaryl, amide, halo, and urea, wherein the alkyl, alkenyl, heteroalkyl, cycloalkyl, heterocycloalkyl, aryl, heteroaryl, and amide are optionally substituted with one or more substituents selected from the group consisting of (C₁-C₁₀)-alkyl, (C₁-C₁₀)-alkenyl, cycloalkyl, heterocycloalkyl, aryl, heteroaryl, alkoxy, amide, sulfonamide, urea, halo, cyano, hydroxy, C(O)OR₆, and C(O)R₇, wherein R₆ is selected from the group consisting of hydrogen, alkyl and alkenyl and R₇ is selected from the group consisting of hydrogen, alkyl, alkenyl, heteroalkyl, cycloalkyl, heterocycloalkyl, aryl, heteroaryl and NR₈R₉, wherein R₈ and R₉ are independently selected from the group consisting of hydrogen, alkyl, alkenyl, aryl and heteroaryl; and
wherein R₂ is selected from the group consisting of halo, fluoroalkyl, and cyano; and
wherein A is heteroarylene; and
wherein X and Z are independently selected from the group consisting of C, N, O and S; and
wherein Y is selected from the group consisting of hydrogen, OH, alkoxy, and NR₁₀R₁₁, wherein R₁₀ and R₁₁ are independently selected from the group consisting of hydrogen, alkyl, heteroalkyl, cycloalkyl, heterocycloalkyl, alkenyl, aryl, heteroaryl, amide, sulfonamide, urea, alkylene-aryl, alkylene-heteroaryl, and C(O)R₁₂ wherein R₁₂ is selected from the group consisting of hydrogen, alkyl, alkenyl, heteroalkyl, cycloalkyl, heterocycloalkyl, aryl and heteroaryl and wherein R₁₀ and R₁₁ may optionally form a ring structure, wherein the aryl, heteroaryl, alkylene-aryl and alkylene heteroaryl are optionally substituted with one or more substituents selected from the group consisting of alkyl, alkenyl, alkynyl, halo, and alkynylene-alkylsilane; and n is an integer from 0 to 5; and
administering to the patient a potentiator of the dihydrofolate reductase inhibitor of Formula I and/or a thymine component.

26. The method of claim 25, wherein the compound of Formula I and/or salt thereof is present at a concentration of 50 μg/mL to 300 μg/mL.

27. The method of claim 25, wherein A is pyridinylene.

28-41. (canceled)
